SEQUENCE LISTING (54 sequences)

SEQ ID NO: 1 - DNA, 1666 bp, homo sapiens
atttgctttctctttttcctttcttccggatgagaggctaagccataatagaaagaatggagaattattgattgaccgtctttattctgtgggctctgattctccaatgggaataccaagggatggttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttggaccctctgggcccatcctggccatggtgggtgaagacgctgatctgccctgtcacctgttcccgaccatgagtgcagagaccatggagctgaagtgggtaagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctaatcttcacgtcgaagtgaagggttatgaggatggagggatccatctggagtgcaggtccaccggctggtacccccaaccccaaatacagtggagcaacgccaagggagagaacatcccagctgtggaagcacctgtggttgcagatggagtgggcctatatgaagtagcagcatctgtgatcatgagaggcggctccggggagggtgtatcctgcatcatcagaaattccctcctcggcctggaaaagacagccagcatttccatcgcagaccccttcttcaggagcgcccagccctggatcgcagccctggcagggaccctgcctatcttgctgctgcttctcgccggagccagttacttcttgtggagacaacagaaggaaataactgctctgtccagtgagatagaaagtgagcaagagatgaaagaaatgggatatgctgcaacagagcgggaaataagcctaagagagagcctccaggaggaactcaagaggaaaaaatccagtacttgactcgtggagaggagtcttcgtccgataccaataagtcagcctgatgctctaatggaaaaatggccctcttcaagcctggtgaggaaatgcttcagatgaggctccaccttgttaaataaattggatgtatggaaaaatagactgcagaaaaggggaactcatttagctcacgagtggtcgagtgaagattgaaaattaacctctgagggccagcacagcagctcatgcctgtaatcctagcactttggaaggctgaggagggcggatcacaaggtcaggagatcaagaccatcctggctaacacggtgaaaccccgtctctactaaaaatacaaaaaataaaaaattagccgggcatggtgacgggcacctgtagtcccagctactcgggaggctgaggcaggagaatggcatgaacccggaaggcagagcttgcagtgagccgagatcacgccactgcactccagcctgggagacagagcgagactctgtctcaagaaaaaaaaaaaaaaaaaaaaa

SEQ ID NO: 2 - protein, 318 aa, homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKSST

SEQ ID NO: 3 - DNA, 2634 bp, homo sapiens
ggcacgaggctgagtgtccgtctcgcgcccggaagcgggcgaccgccgtcagcccggaggaggaggaggaggaggaggaggagggggcggccatggggctgctgtcccagggctcgccgctgagctgggaggaaaccaagcgccatgccgaccacgtgcggcggcacgggatcctccagttcctgcacatctaccacgccgtcaaggaccggcacaaggacgttctcaagtggggcgatgaggtggaatacatgttggtatcttttgatcatgaaaataaaaaagtccggttggtcctgtctggggagaaagttcttgaaactctgcaagagaagggggaaaggacaaacccaaaccatcctaccctttggagaccagagtatgggagttacatgattgaagggacaccaggacagccctacggaggaacaatgtccgagttcaatacagttgaggccaacatgcgaaaacgccggaaggaggctacttctatattagaagaaaatcaggctctttgcacaataacttcatttcccagattaggctgtcctgggttcacactgcccgaggtcaaacccaacccagtggaaggaggagcttccaagtccctcttctttccagatgaagcaataaacaagcaccctcgcttcagtaccttaacaagaaatatccgacataggagaggagaaaaggttgtcatcaatgtaccaatatttaaggacaagaatacaccatctccatttatagaaacatttactgaggatgatgaagcttcaagggcttctaagccggatcatatttacatggatgccatgggatttggaatgggcaattgctgtctccaggtgacattccaagcctgcagtatatctgaggccagatacctttatgatcagttggctactatctgtccaattgttatggctttgagtgctgcatctcccttttaccgaggctatgtgtcagacattgattgtcgctggggagtgatttctgcatctgtagatgatagaactcgggaggagcgaggactggagccattgaagaacaataactataggatcagtaaatcccgatatgactcaatagacagctatttatctaagtgtggtgagaaatataatgacatcgacttgacgatagataaagagatctacgaacagctgttgcaggaaggcattgatcatctcctggcccagcatgttgctcatctctttattagagacccactgacactgtttgaagagaaaatacacctggatgatgctaatgagtctgaccattttgagaatattcagtccacaaattggcagacaatgagatttaagccccctcctccaaactcagacattggatggagagtagaatttcgacccatggaggtgcaattaacagactttgagaactctgcctatgtggtgtttgtggtactgctcaccagagtgatcctttcctacaaattggattttctcattccactgtcaaaggttgatgagaacatgaaggtagcacagaaaagagatgctgtcttgcagggaatgttttatttcaggaaagatatttgcaaaggtggcaatgcagtggtggatggttgtggcaaggcccagaacagcacggagctcgctgcagaggagtacaccctcatgagcatagacaccatcatcaatgggaaggaaggtgtgtttcctggactgatcccaattctgaactcttaccttgaaaacatggaagtggatgtggacaccagatgtagtattctgaactacctaaagctaattaagaagagagcatctggagaactaatgacagttgccagatggatgagggagtttatcgcaaaccatcctgactacaagcaagacagtgtcataactgatgaaatgaattatagccttattttgaagtgtaaccaaattgcaaatgaattatgtgaatgcccagagttacttggatcagcatttaggaaagtaaaatatagtggaagtaaaactgactcatccaactagacattctacagaaagaaaaatgcattattgacgaactggctacagtaccatgcctctcagcccgtgtgtataatatgaagaccaaatgatagaactgtactgttttctgggccagtgagccagaaattgattaaggctttctttggtaggtaaatctagagtttatacagtgtacatgtacatagtaaagtatttttgattaacaatgtattttaataacatatctaaagtcatcatgaactggcttgtacatttttaaattcttactctggagcaacctactgtctaagcagttttgtaaatgtactggtaattgtacaatacttgcattccagagttaaaatgtttactgtaaatttttgttcttttaaagactacctgggacctgatttattgaaatttttctctttaaaaacattttctctcgttaattttcctttgtcatttcctttgttgtctacattaaatcacttgaatccattgaaagtgcttcaagggtaatcttgggtttctagcaccttatctatgatgtttcttttgcaattggaataatcacttggtcaccttgccccaagctttcccctctgaataaatacccattgaactctgaaaaaaaaaaaaaaaaaaa

SEQ ID NO: 4 - protein, 637 aa, homo sapiens
MGLLSQGSPLSWEETKRHADHVRRHGILQFLHIYHAVKDRHKDVLKWGDEVEYMLVSFDHENKKVRLVLSGEKVLETLQEKGERTNPNHPTLWRPEYGSYMIEGTPGQPYGGTMSEFNTVEANMRKRRKEATSILEENQALCTITSFPRLGCPGFTLPEVKPNPVEGGASKSLFFPDEAINKHPRFSTLTRNIRHRRGEKVVINVPIFKDKNTPSPFIETFTEDDEASRASKPDHIYMDAMGFGMGNCCLQVTFQACSISEARYLYDQLATICPIVMALSAASPFYRGYVSDIDCRWGVISASVDDRTREERGLEPLKNNNYRISKSRYDSIDSYLSKCGEKYNDIDLTIDKEIYEQLLQEGIDHLLAQHVAHLFIRDPLTLFEEKIHLDDANESDHFENIQSTNWQTMRFKPPPPNSDIGWRVEFRPMEVQLTDFENSAYVVFVVLLTRVILSYKLDFLIPLSKVDENMKVAQKRDAVLQGMFYFRKDICKGGNAVVDGCGKAQNSTELAAEEYTLMSIDTIINGKEGVFPGLIPILNSYLENMEVDVDTRCSILNYLKLIKKRASGELMTVARWMREFIANHPDYKQDSVITDEMNYSLILKCNQIANELCECPELLGSAFRKVKYSGSKTDSSN

SEQ ID NO: 5 - DNA, 1191 bp, homo sapiens
gcccaagtatcaagagggagagtgagacttgcctgcttctctggcccctggtcctgtcctgttctccagcatggtgtgtctgaagctccctggaggctcctgcatgacagcgctgacagtgacactgatggtgctgagctccccactggctttggctggggacacccgaccacgtttcttgtggcagcttaagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacatcctggaagacgagcgggccgcggtggacacctactgcagacacaactacggggttggtgagagcttcacagtgcagcggcgagttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggattcctgagctgaaatgcagatgaccacattcaaggaagaaccttctgtcccagctttgcagaatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatgacagcgcctcatcttcaacttttgtgctcccctttgcctaaaccgtatggcctcccgtgcatctgtacctcaccctgtacgacaaacacattacattattaaatgtttctcaaagatggagtt

SEQ ID NO: 6 - protein, 266 aa, homo sapiens [sequence omitted]

SEQ ID NO: 7 - DNA, 599 bp, homo sapiens
ccgactccagctctgacttttttcgcggctctcggcttccactgcagccatgtcactcctcttgctggtggtctcagcccttcacatcctcattcttatactgcttttcgtggccactttggacaagtcctggtggactctccctgggaaagagtccctgaatctctggtacgactgcacgtggaacaacgacaccaaaacatgggcctgcagtaatgtcagcgagaatggctggctgaaggcggtgcaggtcctcaggtgctctccctcattctctgctgtctctccttcatcctgttcatgttccagctctacaccatgcgacgaggaggtctcttctatgccaccggcctctgccagctttgcaccagcgtggcggtgtttactggcgccttgatctatgccattcacgccgaggagatcctggagaagcacccgcgagggggcagcttcggatactgcttcgccctggcctgggtggccttccccctcgccctggtcagcggcatcatctacatccacctacggaagcgggagtgagcgccgcgcctcgctcggctgcccccgccccttccgggcccccctgccgcgcgtcctcc

SEQ ID NO: 8 - protein, 163 aa, homo sapiens
MSLLLLVVSALHILILILLFVATLDKSWWTLPGKESLNLWYDCTWNNDTKTWACSNVSENGWLKAVQVLMVLSLILCCLSFILFMFQLYTMRRGGLFYATGLCQLCTSVAVFTGALIYAIHAEEILEKHPRGGSFGYCFALAWVAFPLALVSGIIYIHLRKRE

SEQ ID NO: 9 - DNA, 709 bp, homo sapiens
agaccaacacagcctggaccaccacagccaccccagcagaggccttatggatatgaccagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactccctcaggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagatttttcctctgcatttacacacac

SEQ ID NO: 10 - protein, 98 aa, homo sapiens
RPTQPGPPQPPQQRPYGYDQIMPKKPAEEGNDSEEVPEASGPQNDGKELCPPGKPTTSEKIHERSGPKRGEHAWTHRLRERKQLVIYEEISDPEEDDE

SEQ ID NO: 11 - DNA, 1579 bp, homo sapiens
gaggaggtgcttgccagacactgggtcatggcagtggtcggtgaagctgcagttgcctagggcagggatggagagagagtctgggcatgaggagagggtctcgggatgtttggctggactagattttacagaaagccttatccaggcttttaaaattactctttccagacttcatctgagactccttcttcagccaacattccttagccctgaatacatttcctatcctcatctttcccttcttttttttcctttcttttacatgtttaaatttaaaccattcttcgtgaccccttttcttgggagattcatggcaagaacgagaagaatgatggtgcttgttaggggatgtcctgtctctctgaactttggggtcctatgcattaaataattttcctgacgagctcaagtgctccctctggtctacaatccctggcggctggccttcatcccttgggcaagcattgcatacagctcatggccctccctctaccataccctccacccccgttcgcctaagctcccttctccgggaatttcatcatttcctagaacagccagaacatttgtggtctatttctctgttagtgtttaaccaaccatctgttctaaaagaagggctgaactgatggaaggaatgctgttagcctgagactcaggaagacaacttctgcagggtcactccctggcttctggaggaaagagaaggagggcagtgctccagtggtacagaagtgagacataatggaatcaggcttcacctccaaggacacctatctaagccattttaaccctcgggattacctagaaaaatattacaagtttggttctaggcactctgcagaaagccagattcttaagcaccttctgaaaaatcttttcaagatattctgcctagacggtgtgaagggagacctgctgattgacatcggctctggccccactatctatcagctcctctctgcttgtgaatcctttaaggagatcgtcgtcactgactactcagaccagaacctgcaggagctggagaagtggctgaagaaagagccagaggcctttgactggtccccagtggtgacctatgtgtgtgatcttgaagggaacagagtcaagggtccagagaaggaggagaagttgagacaggcggtcaagcaggtgctgaagtgtgatgtgactcagagccagccactgggggccgtccccttacccccggctgactgcgtgctcagcacactgtgtctggatgccgcctgcccagacctccccacctactgcagggcgctcaggaacctcggcagcctactgaagccagggggcttcctggtgatcatggatgcgctcaagagcagctactacatgattggtgagcagaagttctccagcctccccctgggccgggaggcagtagaggctgctgtgaaagaggctggctacacaatcgaatggtttgaggtgatctcgcaaagttattcttccaccatggccaacaacgaaggacttttctccctggtggcgaggaagctgagcagacccctgtgatgcctgtgacctcaattaaagcaattcctttgacctgtca

SEQ ID NO: 12 - protein, 264 aa, homo sapiens
MESGFTSKDTYLSHFNPRDYLEKYYKFGSRHSAESQILKHLLKNLFKIFCLDGVKGDLLIDIGSGPTIYQLLSACESFKEIVVTDYSDQNLQELEKWLKKEPEAFDWSPVVTYVCDLEGNRVKGPEKEEKLRQAVKQVLKCDVTQSQPLGAVPLPPADCVLSTLCLDAACPDLPTYCRALRNLGSLLKPGGFLVIMDALKSSYYMIGEQKFSSLPLGREAVEAAVKEAGYTIEWFEVISQSYSSTMANNEGLFSLVARKLSRPL

SEQ ID NO: 13 - DNA, 1526 bp, homo sapiens
gtttttttttttttttttaattgcaagcatatttcttttaatgactccagtaaaattaagcatcaagtaaacaagtggaaagtgacctacacttttaacttgtctcactagtgcctaaatgtagtaaaggctgcttaagttttgtatgtagttggattttttggagtccgaaggtatccatctgcagaaattgaggcccaaattgaatttggattcaagtggattctaaatactttgcttatcttgaagagagaagcttcataaggaataaacaagttgaatagagaaaacactgattgataataggcattttagtggtctttttaatgttttctgctgtgaaacatttcaagatttattgatttttttttttcactttccccatcacactcacacgcacgctcacactttttatttgccataatgaaccgtccagcccctgtggagatctcctatgagaacatgcgttttctgataactcacaaccctaccaatgctactctcaacaagttcacagaggaacttaagaagtatggagtgacgactttggttcgagtttgtgatgctacatatgataaagctccagttgaaaaagaaggaatccacgttctagattggccatttgatgatggagctccaccccctaatcagatagtagatgattggttaaacctgttaaaaaccaaatttcgtgaagagccaggttgctgtgttgcagtgcattgtgttgcaggattgggaagggcacctgtgctggttgcacttgctttgattgaatgtggaatgaagtacgaagatgcagttcagtttataagacaaaaaagaaggggagcgttcaattccaaacagctgctttatttggagaaataccgacctaagatgcgattacgcttcagagataccaatgggcattgctgtgttcagtagaaggaaatgtaaacgaaggctgacttgattgtgccatttagagggaactcttggtacctggaaatgtgaatctggaatattacctgtgtcatcaaagtagtgatggattcagtactcctcaaccactctcctaatgattggaacaaaagcaaacaaaaaagaaatctctctataaaatgaataaaatgtttaagaaaagagaaagagaaaaggaattaattcagtgaaggatgattttgctcctagttttggagtttgaatttctgccaggattgaattattttgaaatctcctgtctttttaaactttttcaaaataggtctctaaggaaaaccagcagaacattagcctgtgcaaaaccatctgtttggggagcacactcttccattatgcttggcacatagatctccctgtggtgggattttttttttccctttttttgtgggggagggttggtggtatatttttcccctcttttttccttcctctcctacatctcccttttcccccgatccaagttgtagatggaatagaagcccttgttgctgtagatgtgcgtgcagtctggcagccttaagcccacctgggcacttttagat

SEQ ID NO: 14 - protein, 166 aa, homo sapiens
MNRPAPVEISYENMRFLITHNPTNATLNKFTEELKKYGVTTLVRVCDATYDKAPVEKEGIHVLDWPFDDGAPPPNQIVDDWLNLLKTKFREEPGCCVAVHCVAGLGRAPVLVALALIECGMKYEDAVQFIRQKRRGAFNSKQLLYLEKYRPKMRLRFRDTNGHCCV

SEQ ID NO: 15 - DNA, 1701 bp, homo sapiens
cggactcaagaagttctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaaaaaaaaaaaaa

SEQ ID NO: 16 - protein, 357 aa, homo sapiens
MVDGTLLLLLSEALALTQTWAGSHSLKYFHTSVSRPGRGEPRFSVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 17 - DNA, 2647 bp, homo sapiens
gggccggagttcctgcagagggagcgtcaaggccctgtgctgctgtccctgggggccagaggggttgcccagcatgcccactggcaggagagagggaactgacccacttgctcctaccagcttctgaaggctccaaagtccggagtgcagaaagccaggaccaagagacaggcagctcaccagggtggacaaatcgccagagatgtggtgcattgtcctgttttcacttttggcatgggtttatgctgagcctaccatgtatggggagatcctgtcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcgttttacggggtttgctgcatactatgttgccacagacataaatgaatgcacagattttgtagatgtcccttgtagccacttctgcaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaatattgaaaccaagagtaatgctcttgatatcatcttccaaactgatctaacagggcaaaaaaagggctggaaacttcgctatcatggagatccaatgccctgccctaaggaagacactcccaattctgtttgggagcctgcgaaggcaaaatatgtctttagagatgtggtgcagataacctgtctggatgggtttgaagttgtggagggacgtgttggtgcaacatctttctattcgacttgtcaaagcaatggaaagtggagtaattccaaactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctcatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagc

SEQ ID NO: 18 - protein, 688 aa, homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 19 - DNA, 2035 bp, homo sapiens
gaattccgggctccggggatgaggtcgcggccggcgggtcccgcgctgttgctgctgctgctcttcctcggagcggccgagtcggtgcgtcgggcccagcctccgcgccgctacaccccagactggccgagcctggattctcggccgctgccggcctggttcgacgaagccaagttcggggtgttcatccactggggcgtgttctcggtgcccgcctggggcagcgagtggttctggtggcactggcagggcgaggggcggccgcagtaccagcgcttcatgcgcgacaactacccgcccggcttcagctacgccgacttcggaccgcagttcactgcgcgcttcttccacccggaggagtgggccgacctcttccaggccgcgggcgccaagtatgtagttttgacgacaaagcatcacgaaggcttcacaaactggccgagtcctgtgtcttggaactggaactccaaagacgtggggcctcatcgggatttggttggtgaattgggaacagctctccggaagaggaacatccgctatggactataccactcactcttagagtggttccatccactctatctacttgataagaaaaatggcttcaaaacacagcattttgtcagtgcaaaaacaatgccagagctgtacgaccttgttaacagctataaacctgatctgatctggtctgatggggagtgggaatgtcctgatacttactggaactccacaaattttctttcatggctctacaatgacagccctgtcaaggatgaggtggtagtaaatgaccgatggggtcagaactcttcctgtcaccatggaggatactataactgtgaagataaattcaagccacagagcttgccagatcacaagtgggagatgtgcaccagcattgacaagttttcctggggctatcgtcgtgacatggcattgtctgatgttacagaagaatctgaaatcatttcggaactggttcagacagtaagtttgggaggcaactatcttctgaacattggaccaactaaagatggactgattgttcccatcttccaagaaaggcttcttgctgttgggaaatggctgagcatcaatggggaggctatctatgcctccaaaccatggcgggtgcaatgggaaaagaacacaacatctgtatggtatacctcaaagggatcggctgtttatgccatttttctgcactggccagaaaatggagtcttaaaccttgaatcccccataactacctcaactacaaagataacaatgctgggaattcaaggagatctgaagtggtccacagatccagataaaggtctcttcatctctctaccccagttgccaccctctgctgtccccgcagagtttgcttggactataaagctgacaggagtgaagtaatcatttgagtgcaagaagaaagaggcgctgctcactgttttcctgcttcagtttttctcttatagtaccatcactataatcaacgaacttctcttctccacccagagatggcttttccaacacattttaattaaaggaactgagtacattaccctgatgtctaaatggaccaaagatctgagatccattgtgattatatctgtatcaggtcagcagaagaaggaactgagcagttgaactctgagttcatcaattctaatatttggaaattatctacaatggaatcttccctctgttctctgataacctacttgcttactcaatgcctttaagccaagtcaccctgttgcctatgggaggaggtggaaggatttggcaagctcaaccacatgctatttagttagcatcagttgtcaccaacagtctttctgcaaagggcaggagagctttgggggaaaggaaaaggcttaccaggctgctatggtcaactcttcagaaattttcagagcaatctaaaagcgccaaaattcgctatgtttacagtgatactattaagaaaatgaatgtgattctgctctgtctttttaagtatgatcaaataaaaaatttgtacatcacaatcatttctacc

SEQ ID NO: 20 - protein, 461 aa, homo sapiens
MRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNSSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 21 - DNA, 5916 bp, homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 22 - protein, 360 aa, homo sapiens [sequence omitted]

SEQ ID NO: 23 - DNA, 21 bp, artificial
  Synthetic oligonucleotide
tccccatcacactcacacgca

SEQ ID NO: 24 - DNA, 21 bp, artificial
  Synthetic oligonucleotide
cccttcccaatcctgcaacac

SEQ ID NO: 25 - DNA, 20 bp, artificial
  Synthetic oligonucleotide
agcgggaaactgccttgaca

SEQ ID NO: 26 - DNA, 20 bp, artificial
  Synthetic oligonucleotide
agggcagggcattggatctc

SEQ ID NO: 27 - DNA, 17 bp, artificial
  Synthetic oligonucleotide
tcgccgctgagctggga

SEQ ID NO: 28 - DNA, 20 bp, artificial
  Synthetic oligonucleotide
ccacctcatcgccccacttg

SEQ ID NO: 29 - DNA, 18 bp, artificial
  Synthetic oligonucleotide
cgccgcgagtccgaggat

SEQ ID NO: 30 - DNA, 42 bp, artificial
  Synthetic oligonucleotide
cccggcctcgctctgattgtacccggcctcgctctgattgta

SEQ ID NO: 31 - DNA, 21 bp, artificial
  Synthetic oligonucleotide
ccccgggaaaaccaactacct

SEQ ID NO: 32 - DNA, 21 bp, artificial
  Synthetic oligonucleotide
tgcccatgttcgtgaaaggtc

SEQ ID NO: 33 - DNA, 20 bp, artificial
  Synthetic oligonucleotide
gcggctctcggcttccactg

SEQ ID NO: 34 - DNA, 20 bp, artificial
  Synthetic oligonucleotide
ccgccttcagccagccattc

SEQ ID NO: 35 - DNA, 21 bp, artificial
  Synthetic oligonucleotide
cccccaaccccaaatacagtg

SEQ ID NO: 36 - DNA, 20 bp, artificial
  Synthetic oligonucleotide
ggccgaggagggaatttctg

SEQ ID NO: 37 - DNA, 20 bp, artificial
  Synthetic oligonucleotide
tggccccactatctatcagc

SEQ ID NO: 38 - DNA, 20 bp, artificial
  Synthetic oligonucleotide
tggacccttgactctgttcc

SEQ ID NO: 39 - DNA, 21 bp, artificial
  Synthetic oligonucleotide
ggcccctggtcctgtcctgtt

SEQ ID NO: 40 - DNA, 20 bp, artificial
  Synthetic oligonucleotide
cccgctccgtcccattgaag

SEQ ID NO: 41 - DNA, 20 bp, artificial
  Synthetic oligonucleotide
gcgctggctcacccctacct

SEQ ID NO: 42 - DNA, 21 bp, artificial
  Synthetic oligonucleotide
gccccagggtgcagagatgtc

SEQ ID NO: 43 - DNA, 20 bp, artificial
  Synthetic oligonucleotide
gcctgacattgattcccatt

SEQ ID NO: 44 - DNA, 20 bp, artificial
  Synthetic oligonucleotide
acccaggtcgaccacagata

SEQ ID NO: 45 - DNA, 20 bp, artificial
  Synthetic oligonucleotide
cacagcctctgacagtggaa

SEQ ID NO: 46 - DNA, 20 bp, artificial
  Synthetic oligonucleotide
tggtatcggacgaagactcc

SEQ ID NO: 47 - DNA, 20 bp, artificial
  Synthetic oligonucleotide
gcctgacattgattcccatt

SEQ ID NO: 48 - DNA, 20 bp, artificial
  Synthetic oligonucleotide
acccaggtcgaccacagata

SEQ ID NO: 49 - DNA, 20 bp, artificial
  Synthetic oligonucleotide
cacagcctctgacagtggaa

SEQ ID NO: 50 - DNA, 20 bp, artificial
  Synthetic oligonucleotide
tggtatcggacgaagactcc

SEQ ID NO: 51 - DNA, 23 bp, artificial
  Synthetic oligonucleotide
tgtttatccccatttttcaggtg

SEQ ID NO: 52 - DNA, 23 bp, artificial
  Synthetic oligonucleotide
tccaaagtgctaggattacaggc

SEQ ID NO: 53 - DNA, 23 bp, artificial
  Synthetic oligonucleotide
tgatgctctaatggaaaaatggc

SEQ ID NO: 54 - DNA, 23 bp, artificial
  Synthetic oligonucleotide
gtgctaggattacaggcatgagc

(19) United States
(12) Patent Application Publication
(10) Pub. No.: US 2009/0123932 A1
(43) Pub. Date: May 14, 2009

(54) QUANTITATIVE TEST TO DETECT DISEASE PROGRESSION MARKERS OF EPITHELIAL OVARIAN CANCER PATIENTS

(76) Inventors: Anne-Marie Mes-Masson, Dollard Des Ormeaux (CA); Veronique Ouellet, St-Jean-Sur-Richelieu (CA); Cecile Le Page, Victoria (CA); Diane Provencher, St-Basile-Le-Grand (CA)

Correspondence Address: GOUDREAU GAGE DUBUC, 2000 MCGILL COLLEGE, SUITE 2200, MONTREAL, QC, H3A 3H3, CA

(21) Appl. No.: 12/267,999

(22) Filed: Nov. 10, 2008

Related U.S. Application Data
(60) Provisional application No. 60/986,632, filed on Nov. 9, 2007.

(51) Int. Cl. C12Q 1/68

(52) U.S. Cl. 435/6

(57) ABSTRACT

The present invention concerns a method of prognosing the risk of early ovarian cancer relapse in a subject having ovarian cancer comprising: a) detecting the level of at least one marker selected from the group consisting of BTF4, GCS and HLA-DRbeta1; and b) comparing the level of the above at least one marker with that of a corresponding control sample, wherein the detection of a lower level of the at least one marker compared to that in the control sample is indicative that the subject is at risk of early cancer relapse. Also provided is a method of stratifying a subject suffering from ovarian cancer based on the expression levels of the disclosed markers and kits for practicing the methods of the present invention.

### CROSS REFERENCE TO RELATED APPLICATIONS

This application claims priority under 35 U.S.C. § 119(e), of U.S. provisional application Ser. No. 60/986,632, filed on Nov. 7, 2007. The above document is incorporated herein in its entirety by reference.

### FIELD OF THE INVENTION

The present invention relates to a method of stratifying subjects having ovarian cancer and to prognose the risk of early ovarian cancer relapse. More particularly, the present invention is concerned with molecular markers to stratify ovarian cancer; to prognose disease progression and accordingly to identify appropriate cancer treatment.

### BACKGROUND OF THE INVENTION

Epithelial ovarian cancer (EOC) is the fifth leading cause of cancer-related death in women and represents the most lethal gynaecological malignancy. It is the most common malignant ovarian tumor, representing 80% of all ovarian malignancies (1). EOCs are thought to originate from either the normal ovarian surface epithelium (OSE) itself or from the crypts and inclusion cysts located in the stroma (1). EOCs are heterogeneous and are designated according to their histological subtype: serous, endometrioid, mucinous, clear cell, Brenner, undifferentiated or mixed (association of two or more sub-types) (2, 3).

Due to its lack of symptoms, this disease is diagnosed at an advanced stage (stage III or IV), when the cancer has already spread to secondary sites. The standard treatment for these patients is surgery and platinum-based/taxan-based chemotherapy, although the disease often progresses even after surgery and becomes resistant to standard chemotherapy in less than 2 years. Consequently, the survival rate of patients with advanced stage ovarian cancer is extremely low (<40%). For patients with invasive EOC, aggressive treatment, such as intraperitoneal chemotherapy, has been shown to be more effective and to increase the survival rate compared to standard treatments. However, due to the high toxicity of these treatments, patient stratification is an important variable when considering such therapeutic options. Up to now, there are no reliable clinical factors that can properly stratify patients who would be best suited for aggressive first line chemotherapy. Accordingly, reliable markers, independent and complementary to clinical factors, are needed for better management of these patients.

More recently, genomic and proteomic analyses have emerged as powerful tools for identifying prognostic cancer markers. A large number of promising candidates have been identified by these techniques for cancers of different origins such as breast, prostate, melanoma, B-cell lymphoma and ovary. With more quantitative, reliable and standardized techniques such as real-time quantitative PCR (RT-q-PCR) for the measurement of RNA levels and ELISA for the measurement of protein levels, candidate genes can be validated and tested for their clinical utility. In contrast to ELISA, RT-q-PCR is not dependent on antibody availability and sensitivity and thus may facilitate the initial validation and eventual use of a greater number of markers.

Thus, there remains a need for the identification of prognosis markers for patient stratification. There also remains a need for the identification of markers to determine the risk of disease progression and cancer relapse.

The present invention refers to a number of documents, the content of which is herein incorporated by reference in their entirety.

### SUMMARY OF THE INVENTION

Accordingly, clinically relevant prognosis markers that could be applied in a molecular prognosis test were identified. As a first step, in view of the lack of reliable markers for EOC diagnosis and prognosis, ovarian tumor RNA was screened for potential markers using an Affymetrix™-based gene expression microarray platform. Supervised analysis was performed to identify differentially expressed genes that stratified patients with early and late cancer relapses. These two groups of tumors were defined according to the length of the progression-free interval since intervention (e.g., surgery): tumors from patients who relapsed within 18 months after surgery formed one group and tumors from patients who relapsed after 24 months or did not relapse formed a second group. In a second step, RT-quantitative-PCR was performed to test a subset of these markers. RNAs showing reproducible and statistically significant differences between early and late relapse patients in two different groups of serous and non-serous EOC tumors were selected. Sensitivity and specificity of the markers were also tested. Finally, Kaplan-Meier and Cox regression models were used to assess association with patient survival.

Among the various markers identified, differential expression of two specific markers, namely BTF4 and HLA-DRbeta1 genes, was validated in a set of 41 serous patients and 18 non-serous patients, whereas GCS was only validated in the serous set. In the serous group, BTF4 and GCS mRNA and, in the non-serous group, BTF4 and HLA-DRbeta1 were strongly associated with poor outcome (p<0.05, log rank test). Cox univariate and multivariate analyses identified BTF4 as a prognosis marker with a higher hazard ratio than clinical parameters such as residual disease, age, stage and grade. In the serous cohort, sensitivity and specificity of the test reached 78% and 86%, respectively for BTF4 and reached 84% and 69%, respectively for GCS. Results were reproducible at 95%.

The present study showed that combined DNA microarray and RT-quantitative-PCR identified quantifiable molecular markers to distinguish between EOC patients who will relapse within 18 months from those who will relapse later than two years after initial intervention (e.g., surgery). This approach offered several advantages. First, it avoided technical bias since a second technique was used to validate the initial results obtained with the microarray. Second, it allowed testing the robustness of candidates proven in an independent patient cohort. Third, RT-quantitative-PCR has proven to be a quantitative and reproducible technique. Fourth, in the case of RNA detection, the method is not dependent on antibodies availability, in contrast to the quantitative techniques based on protein detection such as ELISA, which are nevertheless useful.

Thus, in accordance with a first aspect of the present invention there is provided a method for prognosing the risk of early ovarian cancer relapse in a subject having ovarian cancer comprising: a) detecting the level of at least one marker selected from the group consisting of BTF4; GCS and HLA-DRbeta1 in a sample from said subject; and b) comparing the level of said at least one marker with that of a corresponding control sample, wherein the detection of a lower level of said at least one marker compared to that in the corresponding control sample is indicative that the subject is at risk of early cancer relapse.

In accordance with a second aspect of the present invention there is provided a method of stratifying a subject having ovarian cancer comprising: a) detecting the level of at least one marker selected from the group consisting of BTF4; GCS and HLA-DRbeta1; and b) comparing the level of said marker with that of a corresponding control sample, whereby the results of the detecting step enables the stratification of the subject having ovarian cancer as belonging to a subclass of ovarian cancer.

In another aspect, the present invention is concerned with a method of determining disease-free survival in a subject having ovarian cancer comprising: a) detecting the level of at least one marker selected from the group consisting of BTF4; GCS and HLA-DRbeta1; and b) comparing the level of said at least one marker with that of a corresponding control sample, whereby the results of the detecting step enables the determination of disease-free survivalin said subject.

In a specific embodiment, the above-mentioned ovarian cancer is epithelial ovarian cancer. In another embodiment, the above-mentioned ovarian cancer is serous epithelial ovarian cancer. In another embodiment, the above-mentioned ovarian cancer is non-serous epithelial ovarian cancer. In a related embodiment, the non-serous ovarian cancer is clear cell or endometroid non-serous ovarian cancer.

In another particular embodiment, the above-mentioned marker is BTF4. In another specific embodiment, the above-mentioned marker is GCS. In another specific embodiment, a combination of GCS and BTF4 is detected.

In another embodiment, the above-mentioned ovarian cancer is serous epithelial ovarian cancer and the above-mentioned marker is BTF4 or GCS. In another embodiment, the above-mentioned ovarian cancer is non serous epithelial ovarian cancer and the above-mentioned marker is BTF4 or HLA-DRbeta1.

In an embodiment, the above-mentioned sample is an ovarian biopsy. In another embodiment, said sample is a primary culture of cells derived from an ovarian tumor sample from the subject. In a specific embodiment, the sample is a biopsy from a subject who has undergone cytoreductive surgery to remove cancer cells. In another embodiment, the sample is from a biopsy of metastasis derived from the ovarian tumor. In a particular embodiment, the sample is from a subject who has undergone cytoreductive surgery and chemotherapy treatment. In another embodiment, the sample is from a subject who has received a combination of platinum-based and taxan-based chemotherapy as a first line chemotherapy treatment. In yet another specific embodiment, the platinum-based and taxan-based chemotherapy treatment is a combination of paclitaxel and carboplatin.

In an embodiment, said sample is from a subject having received as a first line chemotherapy treatment a treatment selected from the group consisting of i) carboplatin alone; ii) carboplatin in combination with paclitaxel or docetaxel; iii) cisplatin alone; iv) cisplatin in combination with paclitaxel or docetaxel; (v) gemcitabine alone.

In an embodiment, the above-mentioned method further comprises the step of selecting a treatment in light of the expression level of the marker previously determined.

In an embodiment where said subject having ovarian cancer belongs to the early cancer relapse subclass, an aggressive first line chemotherapy treatment is selected. In a particular embodiment, the aggressive treatment is a treatment under clinical trial. In another particular embodiment, the aggressive treatment is selected from the treatments listed in FIG. 9.

In an embodiment, the above-mentioned method further comprises stratifying the subject in a subclass of ovarian cancer selected from the group consisting of i) early cancer relapse; and ii) late cancer relapse or no cancer relapse.

In an embodiment, the above-mentioned level is an mRNA level. In another embodiment the above-mentioned level is a protein level.

In accordance with another aspect of the present invention, there is provided a kit (or commercial package) for prognosing cancer relapse in a subject having ovarian cancer comprising an oligonucleotide probe or set of primers specific to a transcription product of at least one marker selected from the group consisting of BTF4, GCS and HLA-DRbeta1 and instructions to use the probe or primers to determine the level of expression of said marker and to prognose whether the subject is at risk of early cancer relapse.

In accordance with another aspect of the present invention, there is provided a kit for stratifying a subject having ovarian cancer comprising an oligonucleotide probe or set of primers specific to a transcription product of at least one marker selected from the group consisting of BTF4, GCS and HLA-DRbeta1 and instructions to use the probe or primers to stratify said subject.

In accordance with a related aspect, there is provided a kit for assessing the disease-free survival in a subject having ovarian cancer comprising an isolated oligonucleotide probe or set of primers specific to a transcription product of at least one marker selected from the group consisting of BTF4, GCS and HLA-DRbeta1 and instructions to use the probe or primers to determine the level of expression of said marker and determine disease-free survival.

In another specific embodiment, the above described kits further comprise a container for a nucleotide sample from the subject. In a further embodiment, the kit may comprise one or more of: label, control sample, enzymes, buffers etc. In yet a further embodiment, the kit may comprise additional probes and/or primers specific for one or more additional markers of the present invention. In a further embodiment, the kit may comprise additional probes and/or primers specific for a control sequence for assessing whether the sample comprised a sufficient number of cells or cells of a particular cell type (e.g., ovarian cells) or for assessing sample degradation. Other probes, primers or nucleic acid sequences may be included, which allows for direct quantification of the marker in the sample (e.g., spiked nucleic acid sequences).

In accordance with another aspect of the present invention, there is provided a kit for prognosing the risk of early ovarian cancer relapse in a subject having ovarian cancer comprising one or more antibodies specific to one or more markers selected from the group consisting of BTF4, GCS and HLA-DRbeta1 together with instructions to use the one or more antibodies to predict whether a subject is at risk of early cancer relapse.

In accordance with another aspect of the present invention, there is provided a kit for stratifying a subject having ovarian cancer comprising one or more antibodies specific to one or more markers selected from the group consisting of BTF4, GCS and HLA-DRbeta1 together with instructions to use the one or more antibody to stratify said subject.

In accordance with another aspect of the present invention, there is provided a kit for determining disease-free survival in a subject comprising one or more antibodies specific to one or more markers selected from the group consisting of BTF4, GCS and HLA-DRbeta1 together with instructions to use the one or more antibodies to determine disease-free survival.

In a specific embodiment, the above described kits further comprise a container for a cell sample or protein sample from the subject. In a further embodiment, the kit may comprise one or more of: label, control sample, enzyme, buffer etc. In yet a further embodiment, the kit may comprise one or more additional antibodies specific for one or more additional markers of the present invention. In a further embodiment, the kit may comprise one or more further antibodies for a control polypeptide for assessing whether the sample comprised a sufficient number of cells or cells of a particular cell type (e.g., ovarian cells) or for assessing sample degradation. Other antibodies and polypeptides may be included, which allows for direct quantification of the marker in the sample. The kits may also comprise secondary antibodies specific for the primary antibodies of the present invention.

Other objects, advantages and features of the present invention will become more apparent upon reading of the following non-restrictive descriptions of specific embodiments thereof, given by way of examples only with reference to the accompanying drawings.

### BRIEF DESCRIPTION OF THE DRAWINGS

In the appended drawings:

FIG. 1 shows the identification of candidate prognosis markers by DNA microarray analysis. RNA from ovarian epithelia (n=17) were used to select genes which were differentially expressed between subjects showing early disease progression (i.e., within 18 months after initial surgery) and subjects showing no disease progression in the two years following surgery by three statistical algorithms (see Example 1). Each column represents a sample and each row represents the expression of one gene. Color intensity represents gene expression levels transformed in log 10 in the matrix. Light grey indicates lower than median expression and darker grey represents higher than median expression. Light branches represent subjects with late relapse; dark branches represent subjects with early relapse. The row called “relapse” indicates whether the sample in the column above is from a subject who relapsed early or from a subject who relapsed late.

FIG. 2 shows RT-q-PCR validation of microarray analysis. Black, tumors used in the microarray analysis derived from 7 patients who relapsed after 24 months; gray, tumors used in the microarray analysis derived from 10 patients who relapsed within 18 months after initial surgery. Columns, mean results of two independent experiments in duplicate; bars, SE. Relative fold change was calculated according to the Pfaffl algorithm using ERK1 as an internal control gene and TOV1054D was arbitrarily chosen as the reference sample. Statistical analysis was done by the Mann-Whitney U test.

FIG. 3 shows the RT-q-PCR analysis on 41 invasive serous tumors and association with patient survival. Black, serous tumors derived from 15 patients who did not relapse or who did 24 months after diagnosis; gray, serous tumors derived from 26 patients who relapsed within 18 months from surgery. Relative fold change was calculated according to the Pfaffl algorithm using ERK1 as an internal control gene and TOV1054D was arbitrarily chosen as the reference sample. Mean results of two independent experiments in duplicate are presented. A: BTF4; B: HLA-DRbeta1; and C: GCS. The relative fold change corresponds to the expression of the marker relative to the first sample tested normalized with ERK1 gene expression. Statistical analysis was done using Mann-Whitney U test. Kaplan-Meier DFS Significance (P) is calculated by log-rank test.

FIG. 4 shows Kaplan-Meier disease-free survival (DFS; A) and Overall Survival (OS; B) curves in the 41 serous EOC samples. Significance was calculated by log-rank test. “High” concerns subjects having high levels of marker whereas “low” corresponds to subjects showing low levels of markers.

FIG. 5 shows additional RT-q-PCR analysis on 18 invasive non-serous tumors and association with patient survival. Black, non-serous tumors derived from 10 subjects who did not relapse or who did 24 months after diagnosis; gray, non-serous tumors derived from 8 subjects who relapsed within 18 months from surgery. Relative fold change was calculated according to the Pfaffl algorithm using ERK1 as an internal control gene and TOV1054D was arbitrarily chosen as the reference sample. Mean results of two independent experiments in duplicate are presented (A and B). Kaplan Meier DFS(C and D) and OS (E and F) curves in the same subjects, 18 with EOC. Significance (P) was calculated by the log-rank test.

FIG. 6 shows the allele-specific expression of BTF4 established by sequencing genomic DNA (gDNA) and corresponding cDNA from EOC cell lines (OV90, TOV21G; T1V112D and TOV81D) using the listed primers (SEQ ID NOs:43-54). Regions that contained possible single nucleotide polymorphisms (SNPs) based on the Human Genome Browser (genome.ucsc.edu) were tested. The bolded genotypes represent the alleles present in excess in heterozygous cases based on a review of sequence chromatograms (see FIG. 7).

FIG. 7 shows the chromatograms of genomic DNA (gDNA; left) and cDNA (right) for BTF4 SNPs in EOC cell lines (TOV21G and TOV 81D). The arrows indicate evidence of a deviation from a 50:50 allele ratio (B, D, F).

FIG. 8 shows the detection of BTF4 protein by Western blotting. TOV112D or TOV1946 were transiently transfected for 48 hours with plasmids containing gene encoding for BT3.1 or BTF4/BT3.2.

FIG. 9 shows non-limiting examples of more aggressive chemotherapy treatments currently under clinical trials that may be used when a combination of taxan-based and platinum-based chemotherapy treatments is considered insufficient.

FIG. 10 shows survival and progression analysis done using time after cessation of the first treatment of chemotherapy as the starting point. “high” concerns subjects having high levels of marker whereas “low” corresponds to subjects showing low levels of markers.

### DESCRIPTION OF ILLUSTRATIVE EMBODIMENTS

### Definitions

The articles “a,” “an” and “the” are used herein to refer to one or to more than one (i.e., to at least one) of the grammatical objects of the article.

The terms “including” and “comprising” are used herein to mean, and reused interchangeably with, the phrases “including but not limited to” and “comprising but not limited to”.

The term “such as” is used herein to mean, and is used interchangeably with, the phrase “such as but not limited to”.

The term “having ovarian cancer” defines a subject having been diagnosed with ovarian cancer by either a biopsy or other available diagnostic means or combination of means such as molecular tests and molecular/histological imaging. In the context of assessing risk of relapse, a subject “having ovarian cancer” relates to a subject who has been diagnosed with ovarian cancer but who may have received one or more cancer treatments such as cytoreductive surgery or one or more chemotherapy treatments and who is awaiting to see whether another treatment is required. In the case of assessment of risk prior to first line chemotherapy treatment, the assessment is made on ovarian cells obtained following a biopsy or cytoreductive surgery. In an embodiment, the assessment is made following a first line chemotherapy treatment on cells obtained following a second biopsy or cytoreductive surgery on the ovaries or may be taken from a biopsy of metastasis which have grown from the original ovarian tumor. In a particular embodiment, such chemotherapy treatment is a combination of taxan-based and platinum-based chemotherapy such as a combination of carboplatin and paclitaxel. The term includes any form of ovarian cancer of any grade and any stage.

As used herein, the term “level of a marker” or “expression level of a marker” including “level of BTF4”; “level of GCS” and “level of HLA-DRbeta1” is used to refer to transcription and/or translation products (i.e., transcripts or polypeptides). In a more specific embodiment, “level of a marker” or “expression level of a marker” refers to the quantity of mRNA.

“Specificity” refers to the percentage of subjects who test negative for a specific disease or condition (e.g., no relapse or relapse more than 24 months after surgery) who are correctly identified by a test. The specificity is the number of true negative results divided by the sum of the numbers of true negative plus false positive results. No test has 100% specificity because some people who do not have the disease or condition will test positive for it (false positives). In a particular embodiment, “specificity” refers to the fraction of subjects correctly identified as not having experienced epithelial ovarian cancer relapsing or progressing within 18 months after surgery, based on the detection of the expression level of a marker of the present invention.

“Sensitivity” refers to the percentage of subjects who test positive for a specific disease or condition (e.g., cancer relapse within 18 months, short disease-free survival time, cancer progression, stratification within a particular subclass of cancer, etc.) who are correctly identified by a test. The sensitivity is the number of true positive results divided by the sum of the numbers of true positive plus false negative results. No test has 100% sensitivity because some people who have the disease or condition will test negative for it (false negatives). In a particular embodiment, “sensitivity” is the fraction of subjects correctly identified as having experienced epithelial ovarian cancer relapsing or progressing within 18 months after surgery, based on the detection of expression level of a marker of the present invention.

“Marker” in the context of the present invention refers to, without being so limited, a nucleic acid or a polypeptide (or fragment thereof) which is differentially present in a sample taken from a subject stratified in a particular subclass of ovarian cancer (e.g., progressing within 18 months after surgery; not progressing within 24 months after surgery; etc.) as compared to a corresponding sample taken from a control subject (e.g., a person with a negative diagnosis or undetectable cancer or a subject or a population of subjects with late cancer relapse or no cancer relapse).

As used herein, “control sample” refers to a sample of the same type, that is obtained from the same biological source (e.g., biopsy, body fluid, tissue, etc.) as the tested sample but from a healthy subject or population of subjects (i.e., who is/are not afflicted by ovarian cancer), or a sample from a subject or population of subjects who did not relapse within 24 months from surgery or chemotherapy. The control sample can also be a standard sample that contains the same concentration of the above-mentioned markers that is normally found in a corresponding control sample obtained from a healthy subject (or population of subjects) or from a subject (or population of subjects) who did not relapse 24 months after surgery. For example, there can be a standard control sample for the amounts of BTF4, GCS and/or HLA-DRbeta1 normally found in samples from a healthy subject or population of subjects not suffering from ovarian cancer.

A “threshold value” is the level of expression of a marker above which or below which a specific event is likely to occur (e.g., early cancer relapse; late cancer relapse or no relapse; disease free survival, etc.). For example, a “threshold value” for the predisposition of early cancer relapse (or disease free survival; or early cancer progression) may be defined from a population of subjects which does not suffer from ovarian cancer as the average expression level of a marker (i.e., polynucleotides, polypeptides or fragments thereof) of the present invention in that population plus n standard deviations (or average mean signal thereof). In an embodiment, the above-mentioned threshold expression level for each of the markers is determined by Receiver Operator Curves comparing the concentration of each of the markers in an ovarian cancer-free control population with that in a population of subjects who will suffer from early cancer relapse following surgery. Alternatively, it may be defined from a population of subjects having been previously diagnosed with ovarian cancer who do not relapse or who will relapse after more than 24 months after surgery as the average expression level of a marker of the present invention (i.e., polynucleotides, polypeptides or fragments thereof) for that population plus n standard deviations (or average mean signal thereof). In an embodiment, the above-mentioned threshold expression level for each of the markers is determined by Receiver Operator Curves comparing the concentration of each of the markers in the control population of subjects who do not relapse or who relapse more than 24 months after surgery or chemotherapy with that in a population of subjects who will suffer from early cancer relapse following surgery. In an embodiment, a value below the threshold value is indicative that the subject is likely to relapse within 18 months after surgery.

“Early cancer relapse” or “early cancer progression” in the context of the present invention refers to a subject who has been diagnosed with ovarian cancer and who will relapse within 18 months after surgery. By “relapse” is meant the return of signs and symptoms of cancer after a period of improvement or expected improvement (e.g., after cytoreductive surgery and/or chemotherapy treatment).

“Late cancer relapse” or “late cancer progression” in the context of the present invention refers to a subject who has been diagnosed with ovarian cancer and who will not relapse (i.e., will not show any signs or symptoms associated with the disease) at least 24 months after surgery or 18 months after chemotherapy.

“Cytoreductive surgery” means to surgically “reduce” the number of cancer cells in the subject. The standard management for previously untreated advanced-stage epithelial ovarian cancer is optimum cytoreductive surgery followed by chemotherapy. The goals of surgery are to establish a diagnosis, determine the stage and remove as much cancer as possible.

TABLE 1
Surgical Stages Of Ovarian Cancer

| | | |
|---|---|---|
| Stage | I | Limited to the ovaries |
| | IA | One ovary involved |
| | IB | Both ovaries involved |
| | IC | One or both ovaries involved, but with cancer on |
| | | the surface of an ovary, rupture of an ovarian cyst |
| | | malignant ascites or positive abdominal washings |
| Stage | II | Spread to adjacent pelvic structures |
| | IIA | Spread to uterus or fallopian tubes |
| | IIB | Spread to pelvic peritoneum |
| | IIC | Confined to the pelvis, but with malignant ascites |
| | | or positive abdominal washings |
| Stage | III | Spread to the upper abdomen |
| | IIIA | Microscopic spread to the upper abdomen |
| | IIIB | Cancer nodules less than 2 cm in the abdomen |
| | IIIC | Nodules more than 2 cm, or positive pelvic or |
| | | aortic lymph nodes |
| Stage | IV | Distant spread beyond the abdomen, liver, lung etc. |

The stage is determined at surgery. If there are cancer nodules throughout the abdomen, then it is obviously a stage III cancer. If only one ovary is apparently involved, then there has to be an extensive search for microscopic cancer on the other abdominal structures and in the lymph nodes. An early stage is assigned only after a more advanced stage has been excluded.

In all but the earliest cancers, there is often some cancer remaining after surgery. This is because it spreads throughout the abdomen in little nodules, some are only barely visible and others are too small to see. The surgical goal is not to leave any nodules larger than 1 cm. If the residual is this small or smaller than that, the debulking or cytoreduction is considered to have been optimal. Sometimes this is not possible but a maximum effort should be done to try to achieve this optimal situation. This may require removal of a piece of intestine and even a colostomy in some instances.

In addition to stage, the grade is important. There is a grade designated grade 0. This refers to an epithelial adenocarcinoma of low malignant potential, also called a borderline cancer. These cancers tend to be indolent and, although they may be stage III, not recur for many years even without treatment. Grade I adenocarcinomas are easily identified as being from a glandular origin. Grade III cancers are difficult to identify as glandular; they are also called poorly differentiated. Grade II cancers are intermediate in appearance. Grade I cancers are expected to be the least malignant, grade III, the most malignant.

Chemotherapy is often administered after surgery if it was not possible to remove all the cancer at the operation or if the surgeon feels that there is a risk of tiny (microscopic) cancer cells having been left behind. This is known as adjuvant chemotherapy. About 4-6 sessions of chemotherapy are usually given, which lasts 3-6 months.

Before the surgery, if the surgeon believes that the tumor will be difficult to remove, chemotherapy may be given for a few months. This seeks to shrink the cancer and make the operation easier and more effective. It is known as neo-adjuvant chemotherapy. If the cancer has spread to the liver or beyond the abdomen, chemotherapy is the main treatment used. Chemotherapy is also used if the cancer comes back after surgery. Chemotherapy drugs are sometimes given as tablets or, more usually, by injection into a vein (intravenously). The most commonly used drugs to treat ovarian cancer in the first instance are carboplatin or cisplatin, which may be given with paclitaxel (Taxol®) or docetaxel. Other drugs that may be used are gemcitabine, topotecan, doxorubicin and liposomal doxorubicin (Caelyx®). However, these drugs are generally used for relapsing ovarian cancers resistant to taxan-based and platinum-based chemotherapy.

Intravenous chemotherapy is given as a session of treatment, usually over several hours, but sometimes over a few days. This is followed by a rest period of a few weeks, which allows your body to recover from any side effects of the treatment. Together, the treatment and the rest period are known as a cycle of chemotherapy. The number of cycles you have will depend on the type of cancer and how well the chemotherapy seems to be working.

Chemotherapy may also be given directly into the abdomen through a small tube. This is known as intraperitoneal chemotherapy and is only carried out as part of cancer research trials for the treatment of stage 3 ovarian cancer because of its highly toxic effects. It is generally given alongside intravenous chemotherapy.

“Disease-free survival” (DFS) in the context of the present invention is the length of time after treatment for a specific disease during which a patient survives with no sign of the disease. Disease-free survival may be used in a clinical study or trial to help measure how well a new treatment works. Disease-free survival (DFS) denotes the chances of staying free of disease after a particular treatment for a group of individuals suffering from a cancer. It is the percentage of individuals in the group who are likely to be free of disease after a specified duration of time. Very often, two treatment strategies are compared on the basis of the disease-free survival that is achieved in similar groups of patients. Disease-free survival is often used with the term overall survival when cancer survival is described.

“Minimal residual disease” defines evidence for the presence of residua malignant cells even when so few cancer cells are present that they cannot be found by routine means. Tests for minimal residual disease (MRD) can detect some early tumors. In a patient who has been treated (e.g. following surgery and/or first line chemotherapy treatment), the detection of MRD indicates that treatment is incomplete or that the subject is likely to relapse. MRD can thus distinguish subjects who needs intensive and potentially more toxic therapy from those who do not. The general premise underlying MRD is that knowledge of MRD can effectively guide clinical care and increase cure rates.

“Subject” in the context of the present invention relates to any mammal including a mouse, rat, pig, monkey, horse and pets (e.g., cats and dogs). In a specific embodiment, it refers to a human.

Methods for the Determination of mRNA Expression Levels of Markers

The present invention comprises methods to prognose the risk of early cancer relapse; to stratify subjects; to determine disease-free survival; and to choose optimal treatments which are based on the detection of the expression levels of transcripts or transcription products of markers of the present invention. The present invention therefore encompasses any known methods for such determination including any amplification methods such as RT-PCR; real-time RT-PCR; multiplex RT-PCR as well as northern blots, nuclease protection, plaque hybridization, slot blots and the like.

Amplification methods include any known in vitro procedures for obtaining multiple copies (“amplicons”) of a target nucleic acid sequence or its complement or fragments thereof. In vitro amplification refers to production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification and strand-displacement amplification (SDA). Replicase-mediated amplification uses self-replicating RNA molecules and a replicase such as Qβ-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Another known strand-displacement amplification method does not require endonuclease nicking (Dattagupta et al., U.S. Pat. No. 6,087,133). Transcription-mediated amplification is used in the present invention. Those skilled in the art will understand that the oligonucleotide primer sequences of the present invention may be readily used in any in vitro amplification method based on primer extension by a polymerase (see generally Kwoh et al., 1990, Am. Biotechnol. Lab. 8:14 25; Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., 2000, Molecular Cloning—A Laboratory Manual, Third Edition, CSH Laboratories). As commonly known in the art, the oligonucleotide primers are designed to bind to a complementary sequence under selected conditions.

One non-limiting example of a method to detect the level of mRNA of a marker of the present invention in a sample comprises 1) contacting a sample with at least one oligonucleotide probe or primer that hybridizes to at least one marker selected from the group consisting of BTF4; GCS and HLA-DRbeta1 mRNA; and 2) detecting the level of oligonucleotide probe or primer that hybridizes to said at least one marker. The sample may also be tested to control for the presence of another marker specific for a particular type of cells that should be expressed in the sample (e.g., epithelial ovarian cells). The amount of marker detected can be compared to a specific threshold value and therefrom the 1) stratification of the subject; or 2) risk of experiencing early cancer relapse; or can be determined.

In a related aspect, it is possible to verify the efficiency of nucleic acid amplification and/or detection only by performing external control reaction(s) using highly purified control target nucleic acids added to the amplification and/or detection reaction mixture. Alternatively, the efficiency of nucleic acid recovery from cells and/or organelles and the level of nucleic acid amplification and/or detection inhibition (if present) can be verified and estimated by adding to each test sample control cells or organelles (e.g., a defined number of cells from an ovarian cancer cell line expressing a marker of the present invention such as TOV21G; OV 90; TOV 112D and TOV81D) by comparison with external control reaction(s). To verify the efficiency of both sample preparation and amplification and/or detection, such external control reaction(s) may be performed using a reference test sample or a blank sample spiked with cells, organelles and/or viral particles carrying the control nucleic acid sequence(s). For example, a signal from the internal control (IC) sequences present into the cells, viruses and/or organelles added to each test sample that is lower than the signal observed with the external control reaction(s) may be explained by incomplete lysis and/or inhibition of the amplification and/or detection processes for a given test sample. On the other hand, a signal from the IC sequences that is similar to the signal observed with the external control reaction(s), would confirm that the sample preparation including cell lysis is efficient and that there is no significant inhibition of the amplification and/or detection processes for a given test sample. Alternatively, verification of the efficiency of sample preparation only may be performed using external control(s) analyzed by methods other than nucleic acid testing (e.g., analysis using microscopy, mass spectrometry or immunological assays).

Therefore, in one particular embodiment, the methods of the present invention use purified nucleic acids, ovarian cells (preferably epithelial ovarian cells) or viral particles containing nucleic acid sequences serving as targets for an internal control (IC) in nucleic acid test assays to verify the efficiency of cell lysis and of sample preparation as well as the performance of nucleic acid amplification and/or detection. More broadly, the IC serves to verify any chosen step of the process of the present invention.

IC in PCR or related amplification techniques can be highly purified plasmid DNA either supercoiled, or linearized by digestion with a restriction endonuclease and repurified. Supercoiled IC templates are amplified much less efficiently (about 100 fold) and in a less reproducible manner than linearized and repurified IC nucleic acid templates. Consequently, IC controls for amplification and detection of the present invention are preferably performed with linearized and repurified IC nucleic acid templates when such types of IC are used.

The nucleic acids, cells and/or organelles are incorporated into each test sample at the appropriate concentration to obtain an efficient and reproducible amplification/detection of the IC, based on testing during the assay optimization. The optimal number of control cells added, which is dependent on the assay, is preferentially the minimal number of cells which allows a highly reproducible IC detection signal without having any significant detrimental effect on the amplification and/or detection of the other genetic target(s) of the nucleic acid-based assay. A sample to which is added the purified linearized nucleic acids, cells, viral particles or organelles is generally referred to as a “spiked sample”.

In a related aspect, the present invention also provides isolated oligonucleotides including probes and primers to detect a marker of the present invention as well as to detect other control sequences as described above. In specific embodiments, the isolated oligonucleotides have no more than 300, or no more than 200, or no more than 100, or no more than 90, or no more than 80, or no more than 70, or no more than 60, or no more than 50, or no more than 40, or no more than 30 nucleotides. In specific embodiments, the isolated oligonucleotides have at least 12, or at least 13, or at least 14, or at least 15, or at least 17, or at least 18, or at least 19, or at least 20, or at least 30, or at least 40 nucleotides. In other specific embodiments, the isolated oligonucleotides have at least 20 and no more than 300 nucleotides. In other specific embodiments, the isolated oligonucleotides have at least 20 and no more than 200 nucleotides. In other specific embodiments, the isolated oligonucleotides have at least 20 and no more than 100 nucleotides. In other specific embodiments, the oligonucleotides have at least 20 and no more than 90 nucleotides. In other specific embodiments, the isolated oligonucleotides have at least 20 and no more than 80 nucleotides. In other specific embodiments, the isolated oligonucleotides have at least 20 and no more than 70 nucleotides. In other specific embodiments, the isolated oligonucleotides have at least 20 and no more than 60 nucleotides. In other specific embodiments, the isolated oligonucleotides have at least 20 and no more than 50 nucleotides. In other specific embodiments, the isolated oligonucleotides have at least 20 and no more than 40 nucleotides. In other specific embodiments, the isolated oligonucleotides have at least 17 and no more than 40 nucleotides. In other specific embodiments, the isolated oligonucleotides have at least 20 and no more than 30 nucleotides. In other specific embodiments, the isolated oligonucleotides have at least 17 and no more than 30 nucleotides. In other specific embodiments, the isolated oligonucleotides have at least 30 and no more than 300 nucleotides. In other specific embodiments, the isolated oligonucleotides have at least 30 and no more than 200 nucleotides. In other specific embodiments, the isolated oligonucleotides have at least 30 and no more than 100 nucleotides. In other specific embodiments, the isolated oligonucleotides have at least 30 and no more than 90 nucleotides. In other specific embodiments, the isolated oligonucleotides have at least 30 and no more than 80 nucleotides. In other specific embodiments, the isolated oligonucleotides have at least 30 and no more than 70 nucleotides. In other specific embodiments, the isolated oligonucleotides have at least 30 and no more than 60 nucleotides. In other specific embodiments, the isolated oligonucleotides have at least 30 and no more than 50 nucleotides. In other specific embodiments, the isolated oligonucleotides have at least 30 and no more than 40 nucleotides. It should be understood that in real-time PCR, primers also constitute probes without the traditional meaning of this term. Primers or probes appropriate to detect markers of the present invention such as BTF4 (SEQ ID NO:1), GCS (SEQ ID NO:3) and HLA-DRbeta1 (SEQ ID NO:5) in the methods of the present invention can be designed with known methods using sequences distributed across the marker nucleotide sequence (Buck et al. Design Strategies and Performance of Custom DNA Sequencing primers. Biotechniques 27:528-536 (September 1999)). Non limiting examples of primers that may be used in accordance with the present invention include those as set forth in SEQ ID NOs:23-54.

Probes and primers of the invention can be utilized with naturally occurring sugar phosphate backbones as well as modified backbones including phosphorothioates, dithionates, alkyl phosphonates and a nucleotides and the like. Modified sugar phosphate backbones are generally known (Miller, 1988. Ann. Reports Med. Chem. 23:295; Moran et al., 1987. Nucleic Acids Res., 14:5019.). Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA) and Northern blots (RNA detection) as well as in PCR and microarrays. Although less preferred, labeled proteins could also be used to detect a particular nucleic acid sequence to which they bind. Other detection methods include kits containing probes on a dipstick setup and the like.

As used herein the terms “detectably labeled” refer to a marking of a probe or antibody in accordance with the present invention that will allow the detection of the marker expression in methods and kits of the present invention. Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook, J., Fritsch, E. F. & Maniatis, T. (1989). Molecular Cloning: A Laboratory Manual, Cold Spring Harbor Laboratory Press, Cold Spring Harbor, N.Y.). Non-limiting examples of labels include ³H, ¹⁴C, ³²P, and ³⁵S. Non limiting examples of detectable markers include ligands, fluorophores, chemiluminescent agents, enzymes and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe or antibody.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g., uniformly labeled DNA probe using random oligonucleotide primers in low-melt gels), using the SP6/T7 system to transcribe a DNA segment in the presence of one or more radioactive NTP, and the like.

The present invention also relates to arrays. As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips or other solid supports.

As used herein “array of oligonucleotides” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

As used herein “solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres or other geometric configurations.

Any known nucleic acid arrays can be used in accordance with the present invention. For instance, such arrays include those based on short or longer oligonucleotide probes or primers as well as cDNAs or polymerase chain reaction (PCR) products (Lyons P., 2003. Advances in spotted microarray resources for expression profiling. Briefings in Functional Genomics and Proteomics 2, 21-30). Other methods include serial analysis of gene expression (SAGE), differential display, (Ding G. and Cantor C. R., 2004. Quantitative analysis of nucleic acids—the last few years of progress. J Biochem Biol 37, 1-10) as well as subtractive hybridization methods (Scheel J., Von Brevern M. C., Horlein A., Fisher A., Schneider A., Bach A. 2002. Yellow pages to the transcriptome. Pharmacogenomics 3, 791-807), differential screening (DS), RNA arbitrarily primer (RAP)-PCR, restriction endonucleolytic analysis of differentially expressed sequences (READS), amplified restriction fragment-length polymorphisms (AFLP).

“Stringent hybridization conditions” and “stringent hybridization wash conditions” in the context of nucleic acid hybridization experiments such as Southern and Northern hybridization are sequence dependent, and are different under different environmental parameters. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tm can be approximated from the equation of Meinkoth and Wahl, 1984; Tm 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1° C. for each 1% of mismatching; thus, Tm, hybridization and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tm for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see Sambrook, J., Fritsch, E. F. & Maniatis, T. (1989). Molecular Cloning: A Laboratory Manual, Cold Spring Harbor Laboratory Press, Cold Spring Harbor, N.Y. for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of low stringency wash for a duplex of, e.g., more than 100 nucleotides, is 4 6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tm for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Washing with a solution containing tetramethylammonium chloride (TeMAC) could allow the detection of a single mismatch using oligonucleotide hybridization since such mismatch could generate a 10° C. difference in the annealing temperature. The formulation to determine the washing temperature is Tm (° C.)=−682 (L−1)+97 where L represents the length of the oligonucleotide that will be used for the hybridization.

### Methods for the Determination of Protein Expression Levels of Markers

The present invention also comprises methods to prognose the risk of early cancer relapse; to stratify subjects; to determine disease-free survival; and to choose optimal treatments which are based on the detection of the expression levels of protein or translation products of markers of the present invention (e.g., BTF4 (SEQ ID NO:2); GCS (SEQ ID NO:4) and HLA-DRbeta1 (SEQ ID NO:6)) in a sample from a subject having ovarian cancer. The present invention therefore encompasses any known method for such determination.

The terms “polypeptide,” “peptide” and “protein” are used interchangeably herein to refer to a polymer of amino acid residues. The terms apply to amino acid polymers in which one or more amino acid residues are artificial chemical mimetics of corresponding naturally occurring amino acids, as well as to naturally occurring amino acid polymers, those containing modified residues and non-naturally occurring amino acid polymers.

Methods to measure polypeptide expression levels of the markers of this invention include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry and assays based on a property of the protein including but not limited to DNA binding, ligand binding, or interaction with other protein partners.

In an embodiment, the expression level of the above-mentioned markers is determined using an immunoassay. In a specific embodiment, the assay is an ELISA.

An immunoassay is an assay that uses an antibody to specifically bind an antigen (e.g., a marker of the present invention such as BTF4; GCS and HLA-DRbeta1). The immunoassay is characterized by the use of specific binding properties of a particular antibody to isolate, target, and/or quantify the antigen. The phrase “specifically binds” to an antibody or “specifically immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse or human can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

Accordingly, for antibody based methods, both monoclonal and polyclonal antibodies directed to a marker of the present invention are included within the scope of this invention as they can be produced by well established procedures known to those of skill in the art. Additionally, any secondary antibodies, either monoclonal or polyclonal, directed to the first antibodies would also be included within the scope of this invention.

As used herein, the expression “marker antibody” (e.g., “BTF4 antibody”; “GCS antibody”; “HLA-DRbeta1 antibody”) or “immunologically specific anti-marker antibody” (e.g., “immunologically specific anti-BTF4 antibody”; “immunologically specific anti-GCS antibody”; and “immunologically specific anti-HLA-DRbeta1 antibody”) refers to an antibody that specifically binds to (interacts with) a protein marker of the present invention and displays no substantial binding to other naturally occurring proteins other than the ones sharing the same antigenic determinants as the protein marker in question (e.g., BTF4, GCS and HLA-DRbeta1). The term antibody or immunoglobulin is used in the broadest sense, and covers monoclonal antibodies (including full length monoclonal antibodies), polyclonal antibodies, multispecific antibodies and antibody fragments so long as they exhibit the desired biological activity. Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies and multispecific antibodies formed from antibody fragments. Antibody fragments can also refer to binding moieties comprising CDRs or antigen binding domains including, but not limited to, VH regions (VH, VH-VH), anticalins, PepBodies™, antibody-T-cell epitope fusions (Troybodies) or Peptibodies. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The term antibody encompasses herein polyclonal, monoclonal antibodies and antibody variants such as single-chain antibodies, humanized antibodies, chimeric antibodies and immunologically active fragments of antibodies (e.g., Fab and Fab′ fragments) which inhibit or neutralize their respective interaction domains in Hyphen and/or are specific thereto.

Polyclonal antibodies are preferably raised in animals by multiple subcutaneous (sc), intravenous (iv) or intraperitoneal (ip) injections of the relevant antigen with or without an adjuvant. It may be useful to conjugate the relevant antigen to a protein that is immunogenic in the species to be immunized, e.g., keyhole limpet hemocyanin, serum albumin, bovine thyroglobulin or soybean trypsin inhibitor using a bifunctional or derivatizing agent such as, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups.

Animals may be immunized against the antigen, immunogenic conjugates or derivatives by combining the antigen or conjugate (e.g., 100 μg for rabbits or 5 μg for mice) with 3 volumes of Freund's complete adjuvant and injecting the solution intradermally at multiple sites. One month later, the animals are boosted with the antigen or conjugate (e.g., with ⅕ to 1/10 of the original amount used to immunize) in Freund's complete adjuvant by subcutaneous injection at multiple sites. Seven to 14 days later, the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates can also be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293 (see also, e.g., Lindenbaum et al., 2004).

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin and thymidine (HAT medium), which substances prevent the growth of HGPRT-deficient cells.

Non-limiting examples of specific antibodies that bind selectively to BTF4 (SEQ ID NO:2) that may be used in accordance with the methods of the present invention include commercially available BTF4 antibodies from Strategic Diagnostics and from Novus Biological.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead or a microbead. The sample is preferably a biological fluid sample taken from a subject. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horseradish peroxidase, alkaline phosphatase and others commonly used in an ELISA), and calorimetric labels such as colloidal gold or colored glass or plastic beads. Alternatively, the marker in the sample can be detected using an indirect assay, wherein, for example, a second labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

If desired, the sample can be prepared to enhance detectability of the markers. For example, to increase the detectability of markers, a sample from the subject can be fractionated by, e.g., Cibacron™ blue agarose chromatography and single stranded DNA affinity chromatography, anion exchange chromatography, affinity chromatography (e.g., with antibodies) and the like. The method of fractionation depends on the type of detection method used. Any method that enriches for the protein of interest can be used. Sample preparations, such as pre-fractionation protocols, are optional and may not be necessary to enhance detectability of markers depending on the methods of detection used. For example, sample preparation may be unnecessary if antibodies that specifically bind markers are used to detect the presence of markers in a sample.

Typically, sample preparation involves fractionation of the sample and collection of fractions determined to contain the markers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis. Examples of methods of fractionation are described in WO/2003/057014.

### Methods for the Determination of Optimal Cancer Treatments

As indicated above, the methods of the present invention allow for the identification of subjects who suffer from a form of ovarian cancer which is likely to relapse or progress within 18 months after surgery or chemotherapy and those who will not relapse or who will relapse more than 24 months after surgery or 18 months after chemotherapy. It also allows for the stratification of cancer subjects, in particular into subclasses of ovarian cancer. A more reliable determination of the risk of progression or cancer relapse enables to 1) select the best therapeutic treatment or combination of treatments available for more aggressive forms of cancer; and 2) select the less toxic and invasive treatment for subjects afflicted with a form of ovarian cancer that is more responsive to taxan-based and platinum-based treatments and which is less likely to progress after surgery and/or chemotherapy. Thus, the methods of the present invention allow for optimal treatment management of subjects suffering from more aggressive forms of cancer, and limit unnecessary highly toxic treatments in subjects who do not need such treatments.

In an aspect, the present invention is concerned with a method of stratifying a subject having ovarian cancer comprising: a) detecting the level of at least one marker selected from the group consisting of BTF4; GCS and HLA-DRbeta1 and b) comparing the level of the marker with that of a corresponding control sample, whereby the results of the detecting step enables the stratification of the subject having ovarian cancer as belonging to a subclass of ovarian cancer. In an embodiment, the subclasses are i) early cancer relapse; and ii) late cancer relapse or no cancer relapse. The detection of a lower level of BTF4, GCS and/or HLA-DRbeta1 compared to that in the corresponding control sample is indicative that the subject is at risk of early cancer relapse. The detection of a higher level of BTF4, GCS and/or HLA DRbeta1 compared to that in the corresponding control sample is indicative that the subject will not relapse within 24 months from surgery or treatment. In an embodiment, the risk of cancer relapse or progression is assessed following surgery. In another embodiment, the risk of cancer relapse or progression is assessed following first line chemotherapy. In a further embodiment, the risk of cancer relapse or progression is assessed following second line or third line chemotherapy.

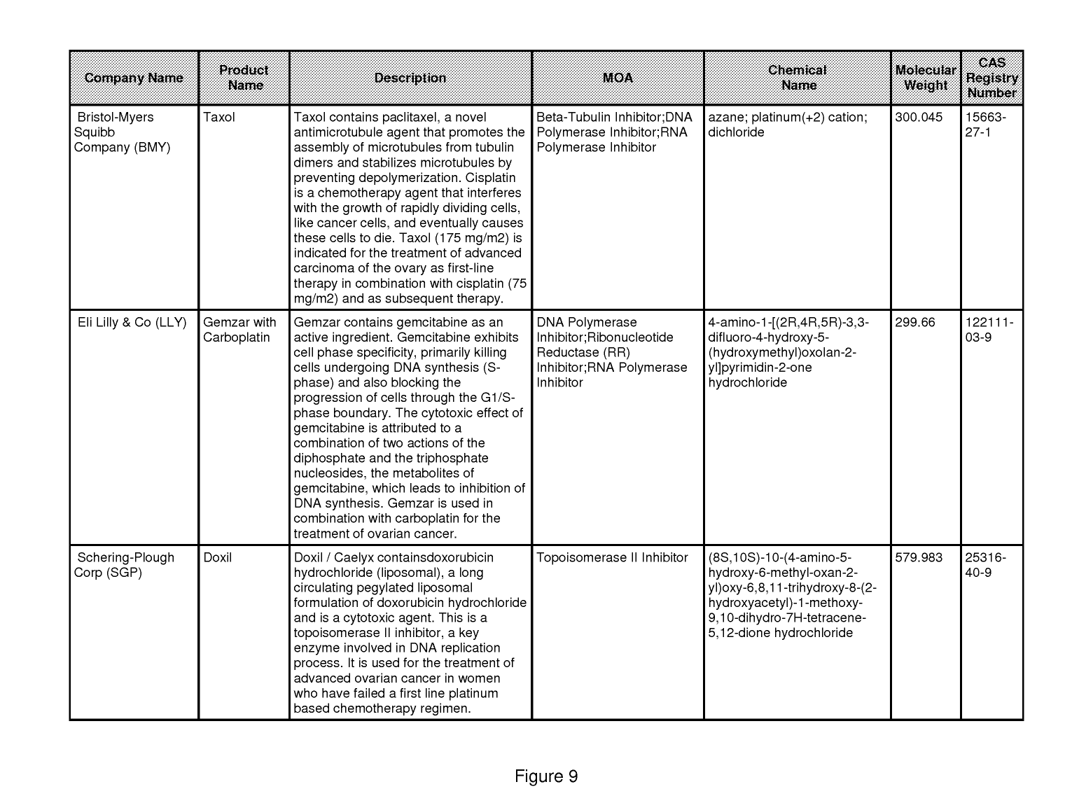

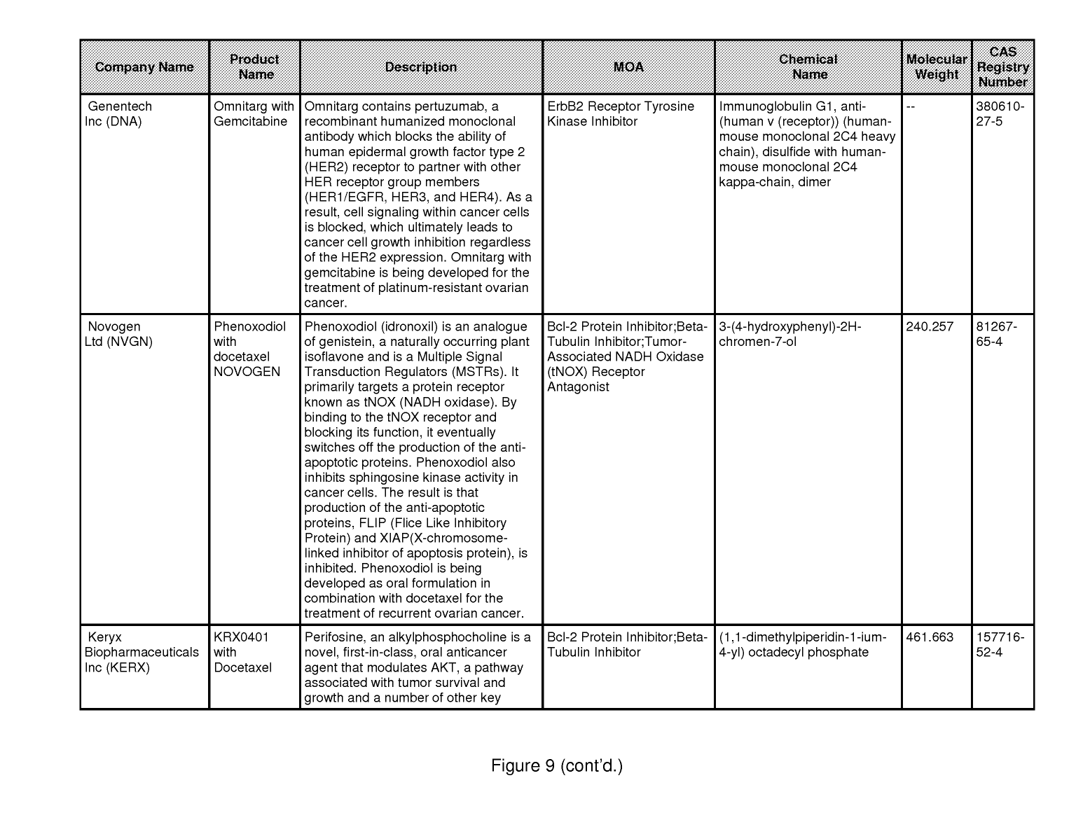

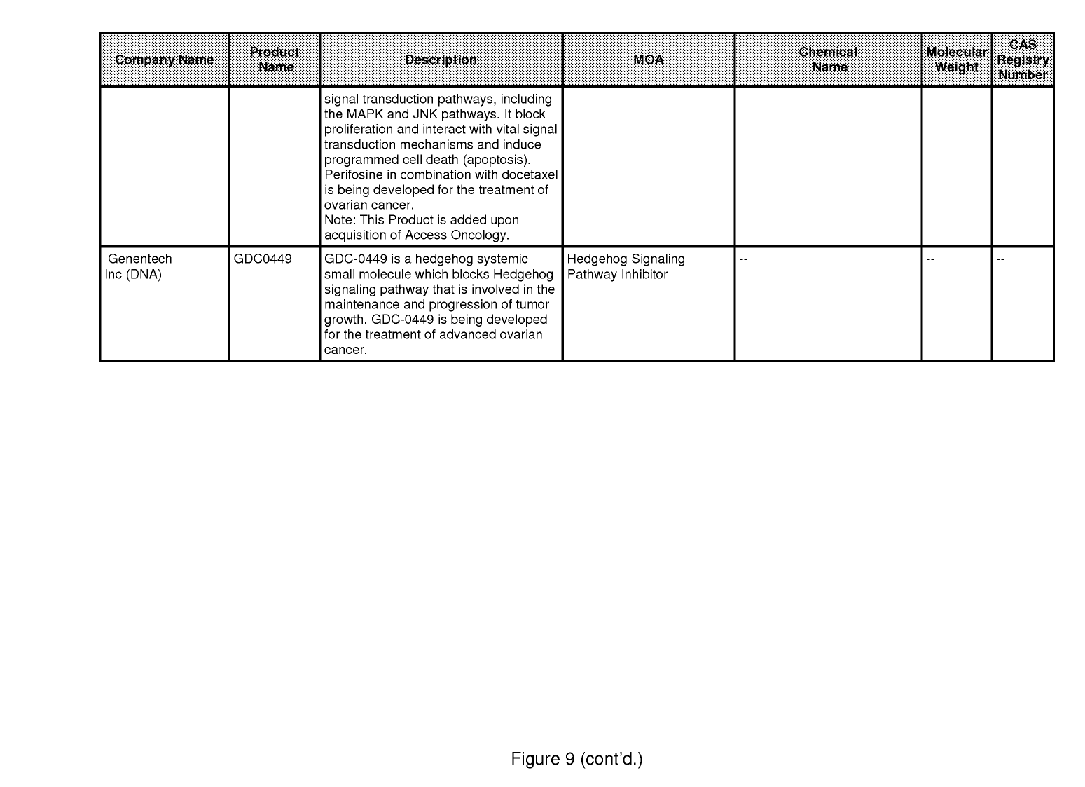

When a subject is stratified as belonging to the subclass of cancer subjects who will relapse within 18 months from treatment, a more aggressive chemotherapy treatment will be selected. For example, the subject may be given intraperitoneal chemotherapy in addition to intravenous injection of taxan-based and platinum-based treatments (e.g., combination of paclitaxel and carboplatin; combination of docetaxel and carboplatin; combination of cisplatin and paclitaxel). Non-limiting examples of additional more aggressive chemotherapy treatments that may be selected include: topotecan, doxorubicin; liposomal doxorubicin, gemcitabine and Omnitarg™; docetaxel and phenoxodiol; docetaxel and perifosine and GDC0449 (an orally-administered small molecule Hedgehog antagonist, as a maintenance therapy for ovarian cancer patients in second or third complete remission) (see FIG. 9).

The present invention is illustrated in further details by the following non-limiting examples.

### EXAMPLE 1

### Materials and Methods

Patients and tissue specimens. Serous tumor samples from 177 chemotherapy naïve patients were collected and banked in liquid nitrogen following appropriate consent from patients undergoing surgery within the Division of Gynaecologic Oncology at the Centre hospitalier de l'Université de Montreal (CHUM) from 1995 to 2004. An independent pathologist scored tumor grade and a gynecologist oncologist scored tumor stage and residual disease according to criteria from the International Federation of Gynecology and Obstetrics (FIGO). Clinical data on survival and progression-free interval were defined according to Response Evaluation Criteria in Solid Tumors Criteria (RECIST) 5) criteria. Good quality RNA samples from the bank, as monitored by 2100 Bioanalyzer™ (Agilent Technologies, Mississauga, ON, Canada) were used for this analysis. Subject survival was calculated from the time of diagnosis until the first progression. For the microarray study, RNA was purified from samples collected between 1995 and 2002. The majority of samples were excluded based on inappropriate histopathology, incomplete follow-up, preoperative chemotherapy or insufficient material. Less than 10% were excluded on RNA quality, and this was not correlated to age of sample. RNAs used for hybridization to the Affymetrix™ HuFL arrays were selected based on sufficient quantity and an RNA integrity number score of >8.7. In total, 17 samples matched the eligibility criteria for this study. For the RT-q-PCR, 40 independent patients were included based on RNA quality and eligibility criteria. Eligibility criteria for inclusion in the study were as follows: no preoperative treatment, tumors of grade 2 or 3, clinical follow-up of at least 18 months or until death and completed informed consent. All patients received a Paclitaxel/Carboplatin chemotherapy as an initial therapy after surgery. A single gynecologic oncologist reviewed the clinical data for all patients. The characteristics of the tumors and patient outcome for the sample sets are summarized in Table 2. The characteristics of the tumors and patient outcome for the sample sets are summarized in Table 2.

TABLE 2
Samples description

| | | | Number of |
|---|---|---|---|
| | Sample properties | | samples |
| Microarray analysis | Histopathology type | serous | 17 |
| samples | Grade | 2 | 6 |
| | | 3 | 11 |
| | Stage | 1 | 1 |
| | | 2 | 2 |
| | | 3 | 14 |
| | Residual disease | unknown | 5 |
| | | <2 cm | 6 |
| | | >2 cm | 6 |
| | Mean age at diagnosis | 60 | — |
| | (years) | | |
| | Mean survival (months) | 35 | — |
| | Disease free survival | <18 months | 10 |
| | | >24 months | 7 |
| Larger set of | Histopathology type | serous | 41 |
| serous samples | Grade | 2 | 12 |
| | | 3 | 29 |
| | Stage | 1 | 2 |
| | | 2 | 2 |
| | | 3 | 35 |
| | | 4 | 2 |
| | Residual disease | unknown | 4 |
| | | <2 cm | 20 |
| | | >2 cm | 17 |
| | Mean age at diagnosis | 63 | — |
| | (years) | | |
| | Mean survival (months) | 30 | — |
| | Disease free survival | <18 months | 26 |
| | | >24 months | 15 |
| Set of samples | Histopathology type | endometrioid | 8 |
| with different | | clear cell | 9 |
| histopathology | | mucinous | 1 |
| types | Grade | 2 | 7 |
| | | 3 | 11 |
| | Stage | 1 | 5 |
| | | 2 | 1 |
| | | 3 | 11 |
| | Residual disease | <2 cm | 12 |
| | | >2 cm | 6 |
| | Mean age at diagnosis | 54 | — |
| | (years) | | |
| | Mean survival (months) | 34 | — |
| | Disease free survival | <18 months | 8 |
| | | >24 months | 10 |

### RNA Extraction

Total RNA was extracted from homogenized tumor tissue with TRIZol™ reagent (Gibco/BRL, Life Technologies Inc., Grand Island, N.Y., USA). Quality was assessed with a 2100 Bioanalyzer™ with an RNA 6000 Nano LabChip™ kit (Agilent Technologies, Mississauga, ON, Canada) according to the manufacturer's protocol. Linear amplification of RNA was performed with RAMP (Alethia Biotherapeutics, Montreal, Qc, Canada).

### Microarray Analysis

Affymetrix™ HuFL arrays were used to hybridize label targets prepared from total RNA (6,8). Hybridization assays and data collection were undertaken at the McGill University and Genome Quebec Innovation Centre (Montreal, Quebec, Canada) (10). Affymetrix™ raw values were assigned by Affymetrix™ GeneChip™ software (MAS4) with an accompanying reliability score of present (P), marginal (M) and ambiguous (A). No genechip used in this study had >30% A Score. Presence of stromal cells was estimated by detectable expression of CD31 and myosin in the genechip (probes D10667, L34657, and X96783). No genechip used in this study had detectable signals for these probe sets. Global normalization and preprocessing of the data were previously described in detail (7,8). Candidate genes that exhibited statistically significant differences in expression within a tested set were selected using the significance analysis of microarray (9) (1,000 permutations done and false discovery rate of <5%) and the Mann-Whitney U test with the GeneSpring™ software (Agilent Technology; P<0.05 with Benjamini and Hochberg false discovery rate of 5%).

Hybridization assays and data collection were undertaken at the McGill University and Genome Quebec Innovation Centre (Montreal, Qc, Canada). Briefly, GeneChip® expression arrays enable one to measure expression levels of transcripts both quantitatively and qualitatively. Affymetrix™ array technology involves the in-situ synthesis of hundreds of thousands of distinct oligonucleotide sequences onto a glass array using photolithography and combinatorial chemistry. Each 25-mer oligonucleotide is represented by millions of copies in a specific area. Each transcriptional sequence is spanned by 11-20 pairs of oligonucleotide probes randomly spaced throughout the array. All sequences designed on the array are selected from GenBank, dbEST and RefSeq. Target RNA is reverse transcribed into cDNA and in-vitro transcription is performed to generate biotin-labeled cRNA for subsequent hybridization. Hybridized target cRNA is stained with streptavidin phycoerythrin and arrays are scanned using a GeneArray Scanner at an excitation wavelength of 488 nm. Light emissions at 570 nm are proportional to the bound target at each oligonucleotides position on the GeneChip® array. Raw values were assigned by Affymetrix™ GeneChip™ software (MAS4) for each probe set from the scanned image.

Real-Time Quantitative-PCR (q-PCR)

cDNA synthesis was performed from 2 μg of total RNA using the SuperScript™ First-Strand Synthesis System for RT-PCR (Invitrogen Life Technologies, Carlsbad, Calif.) according to the manufacturer's protocol. Q-PCR was performed with a Quantitect™ SYBR Green PCR reagent as described in the manufacturer's instructions (QIAGEN Inc., Mississauga, ON) using a Rotor-Gene™ 3000 Real-Time Centrifugal DNA Amplification System (Corbett Research, Montreal Biotech Inc., Montreal, Qc, Canada). Q-PCR analysis for each gene was performed in duplicate in two independent analyses but when two experiments were concordant, and where the average just failed to reach significance, a third experiment was done and used to determine the average. The Pfaffl method served to evaluate the relative quantity of gene expression with ERK1 gene expression as the internal control. The primer sequences and amplification temperatures used are listed in Table 3 below.

### Statistical Microarray Analyses

Two groups of tumors were defined for each set according to the progression-free interval of the patient: tumors from patients who relapsed within 18 months after surgery formed one group and tumors from patients who did not relapse or relapsed after 24 months formed a second group. Candidate genes that exhibited statistically significant differences in expression between the two groups within a tested set were selected using three methods of analysis: Signal-to-noise, Significance Analysis of Microarray (SAM) (http://www-stat-class.standford.edu/SAM/SAMServlet) (FDR<5%); a student t-test and the Mann-Withney U test using the GeneSpring™ software (Silicon Genetics, Redwood City, Calif.) (p<0.05). Candidates identified in three statistical methods and showing the highest differential expression were selected for further analysis. Clustering (based on Pearson correlation) and class prediction analysis (k-nearest neighbour algorithm) were performed with GeneSpring™ software (Silicon Genetics, Redwood City, Calif.).

TABLE 3
List of primers used for quantitative PCR (q-PCR)

| | | | | Annealing | |
|---|---|---|---|---|---|
| | | | | temperature | Product |
| Gene | Direction | Sequence 5′-3′ | SEQ ID NO | (° C.) | length (pb) |
| PTP4A2 | F | TCCCCATCACACTCACACGCA | 23 | 58 | 350 |
| PTP4A2 | R | CCCTTCCCAATCCTGCAACAC | 24 | | |
| C1r-C1s | F | AGCGGGAAACTGCCTTGACA | 25 | 58 | 190 |
| C1r-C1s | R | AGGGCAGGGCATTGGATCTC | 26 | | |
| GCS | F | TCGCCGCTGAGCTGGGA | 27 | 58 | 130 |
| GCS | R | CCACCTCATCGCCCCACTTG | 28 | | |
| CMH-E | F | CGCCGCGAGTCCGAGGAT | 29 | 58 | 135 |
| CMH-E | R | CCCGGCCTCGCTCTGATTGTA | 30 | | |
| SSX2 | F | CCCCGGGAAAACCAACTACCT | 31 | 58 | 210 |
| SSX2 | R | TGCCCATGTTCGTGAAAGGTC | 32 | | |
| YMP | F | GCGGCTCTCGGCTTCCACTG | 33 | 58 | 220 |
| YMP | R | CCGCCTTCAGCCAGCCATTC | 34 | | |
| BTF4 | F | CCCCCAACCCCAAATACAGTG | 35 | 58 | 173 |
| BTF4 | R | GGCCGAGGAGGGAATTTCTG | 36 | | |
| NNMT | F | TGGCCCCACTATCTATCAGC | 37 | 59 | 184 |
| NNMT | R | TGGACCCTTGACTCTGTTCC | 38 | | |
| HLA-DRbeta1 | F | GGCCCCTGGTCCTGTCCTGTT | 39 | 61 | 185 |
| HLA-DRbeta1 | R | CCCGCTCCGTCCCATTGAAG | 40 | | |
| ERK1 | F | GCGCTGGCTCACCCCTACCT | 41 | 58 | 199 |
| ERK1 | R | GCCCCAGGGTGCAGAGATGTC | 42 | | |

Statistical Analysis for q-PCR

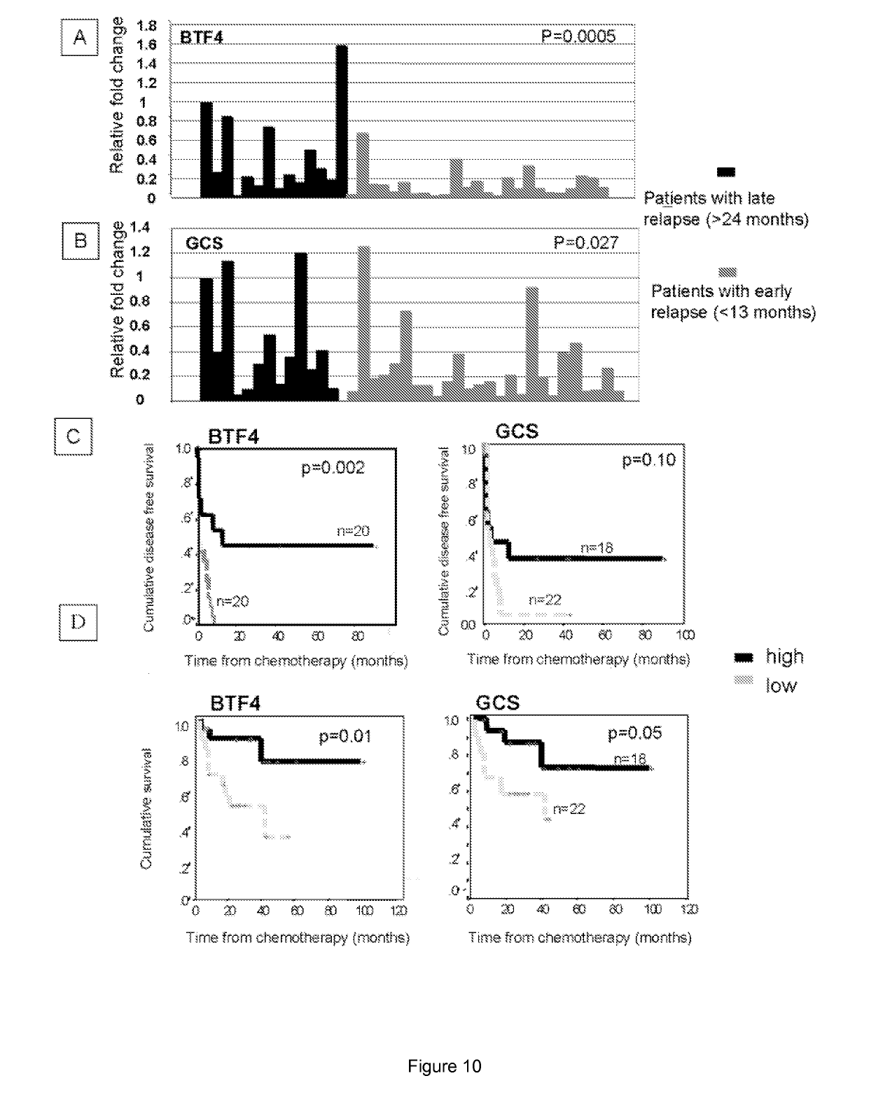

Differential expressions of candidate genes measured by q-PCR were evaluated by a U-test. For survival and progression analyses, the Cox survival model with time-dependent covariates and Kaplan-Meier curves coupled with the log rank test were used. Receiver operating characteristics (ROC) curves were charted for each marker to define a threshold of expression corresponding to the best sensitivity and specificity for patient progression before and after 18 months from initial diagnosis (PLO.05 and area >0.70). For Cox regression analysis markers were treated as categorical variables based on the threshold of expression. Survival and progression analysis done using time after cessation of the first treatment of chemotherapy as the starting point were also performed (see FIG. 10. For HLA-DRbeta1, data not shown). All statistical analyses were performed using the Statistical Package for the Social Sciences (SPSS) software, version 11.0 (SPSS Inc., Chicago, Ill., USA) and statistical significance was set at p<0.05.

### Western Blotting:

Proteins were lysed with cold lysis buffer (10 mM Tris-HCl, pH 7.4, 150 mM NaCl, 1 mM EDTA, 1 mM DTT/1 mM NaF/0.5% NP-40/0.5 mM PMSF/0.2 mM sodium orthovanadate/2 μg/ml of aprotinin, leupeptin and pepstatin), 30 min on ice, then centrifugated at 13000 rpm for 5 min at 4° C. Supernatants containing proteins were collected and measured with Bradford reagent (Biorad). Proletins were then boiled in loading buffer, separated by 10% SDS-PAGE and transferred on a nitrocellulose membrane (Biorad) under refrigerated conditions (60 V, 2 h). The membrane was saturated with 5% milk/PBS/0.1% Tween 20. Immunodetection was done as described in the protocol of the ECL kit (Amersham Pharmacia). Briefly, membranes were incubated overnight at 4° C. with the specific antibody antiCD277 (ebioscience) (1/250 in PBS/Tween/5% milk), washed 2 times with PBS/0.05% Tween 20 and incubated for another 30 min at room temperature with peroxidase conjugated anti-IgG1 antibodies (1/5000, Santa-Cruz Biotechnology Inc.).

### EXAMPLE 2

### Identification of Candidate Genes Related to Disease Progression

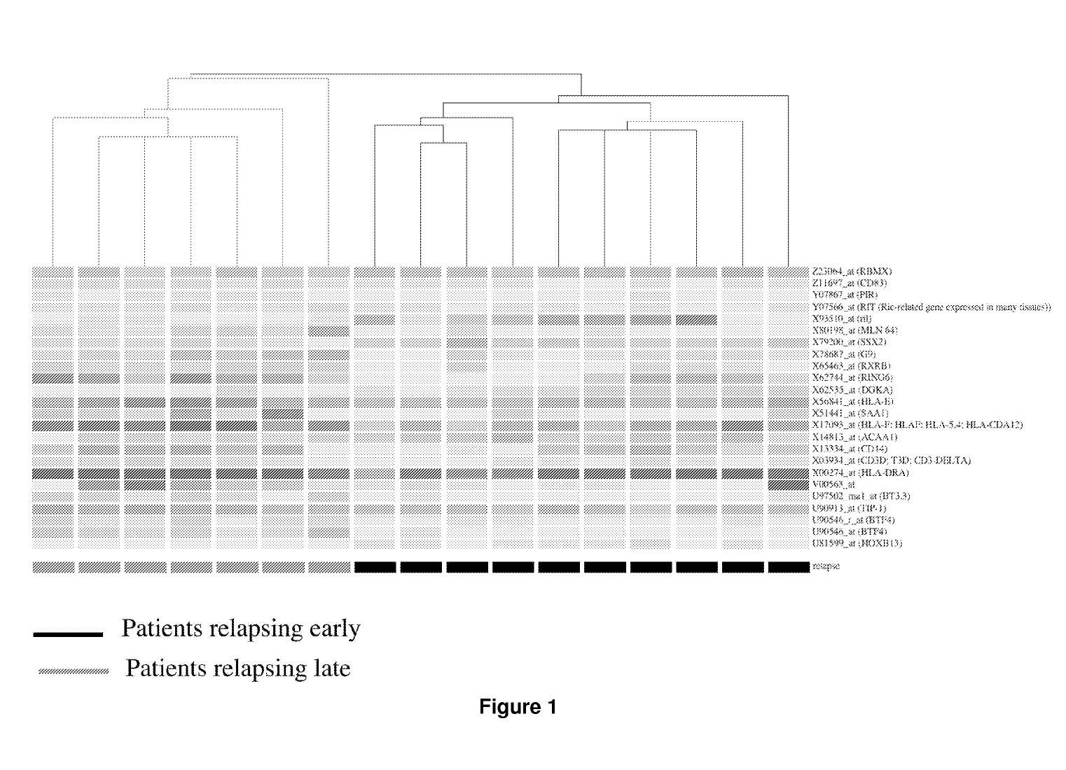

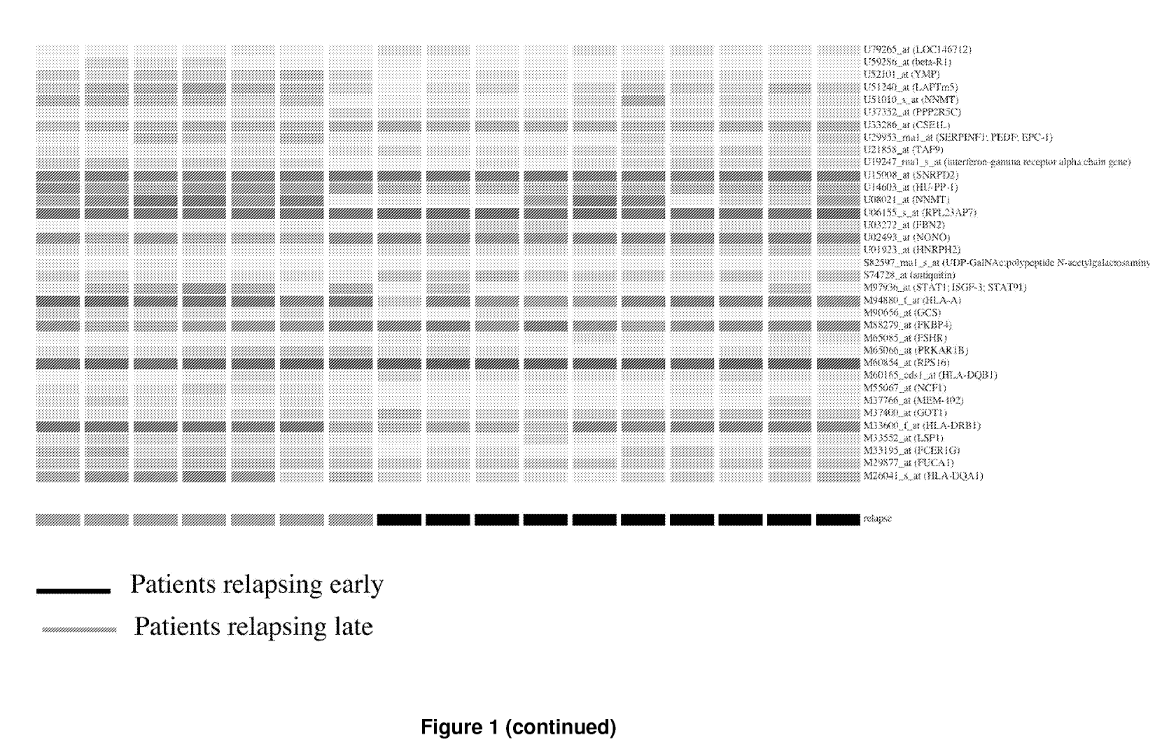

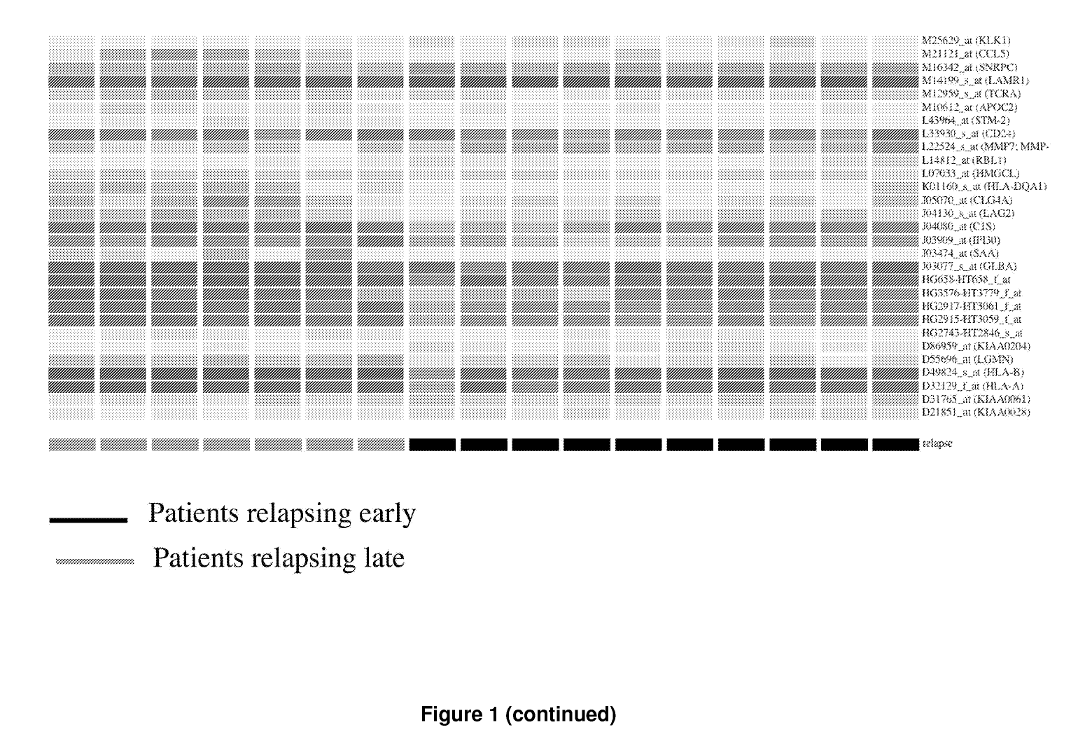

To identify potential genes whose expression could be used as prognosis markers, a supervised microarray analysis was performed using expression profiles generated from serous tumors from ten patients showing early disease progression (within 18 months after surgery) and seven patients who showed no disease progression prior to two years. Using three different statistical methods, a total of 88 differentially expressed genes were identified distinguishing these two groups. In a further analysis, the expression profiles of these 88 genes were used to perform a hierarchical clustering which showed that they correctly separated the two groups of patients (FIG. 1). Using the k-nearest neighbour algorithm, the expression profile of the 88 genes allowed the proper classification of 16 samples (94% accuracy), while one sample exhibiting later relapse unclassified. Since an objective prognosis of ovarian patients based on a limited set of genes would be more appropriate for clinical application, this analysis was refined to candidate genes identified by all three statistical methods (GCS/GCLC (SEQ ID NOs:3 and 4), CMHE1/HLA-E/HLA-6.2 (SEQ ID NOs:16 and 17), BTF4/BTN3A2 (SEQ ID NOs:1 and 2), FUCA1 (SEQ ID NOs:19 and 20), HLA-C and SSX2 (SEQ ID NOs:9 and 10)) or to genes that, in each of the three analyses, presented the most significant differential expression between the two groups and a greater than twofold difference (C1r/C1S, PTP4A2/PTP(CAAX2)/PRL2/HH12/HH7-2 (SEQ ID NOs:13 and 14), YMP/EMP3 (SEQ ID NOs:7 and 8), NNMT (SEQ ID NOs:11 and 12) and HLA-DRbeta1 (SEQ ID NOs:5 and 6)). Since the probe set for HLA-C no longer corresponds to the correct Genbank number, this gene was eliminated from further analysis. Ten genes were thus selected for further quantitative validation.

### EXAMPLE 3

### Validation of Candidates by Real-Time Quantitative PCR

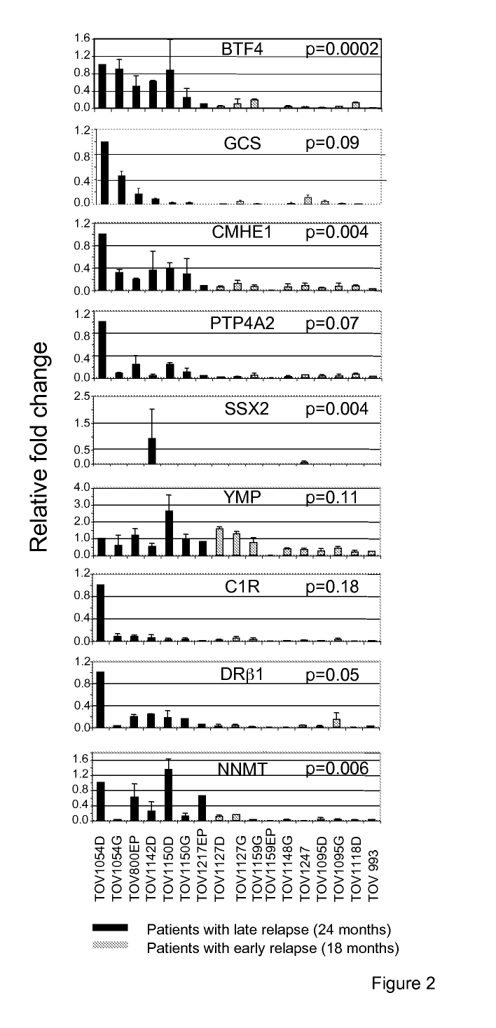

To validate the microarray results by a quantitative technique for RNA measurement, an RT-q-PCR analysis was performed on linearly amplified RNA corresponding to the 17 samples included in the microarray analysis. Statistically significant (p<0.05, U-test) overexpression of BTF4, NNMT, CMHE1 and HLA-DRbeta1 was observed between the two groups (FIG. 2). SSX2 showed expression in only two samples and no FUCA1 expression was detectable by RT-quantitative-PCR. Only the genes presenting significant differential expression or a trend towards significance were chosen for further analysis.

### EXAMPLE 4

### Independent Validation of Candidate Genes on a Larger Set of Serous Samples

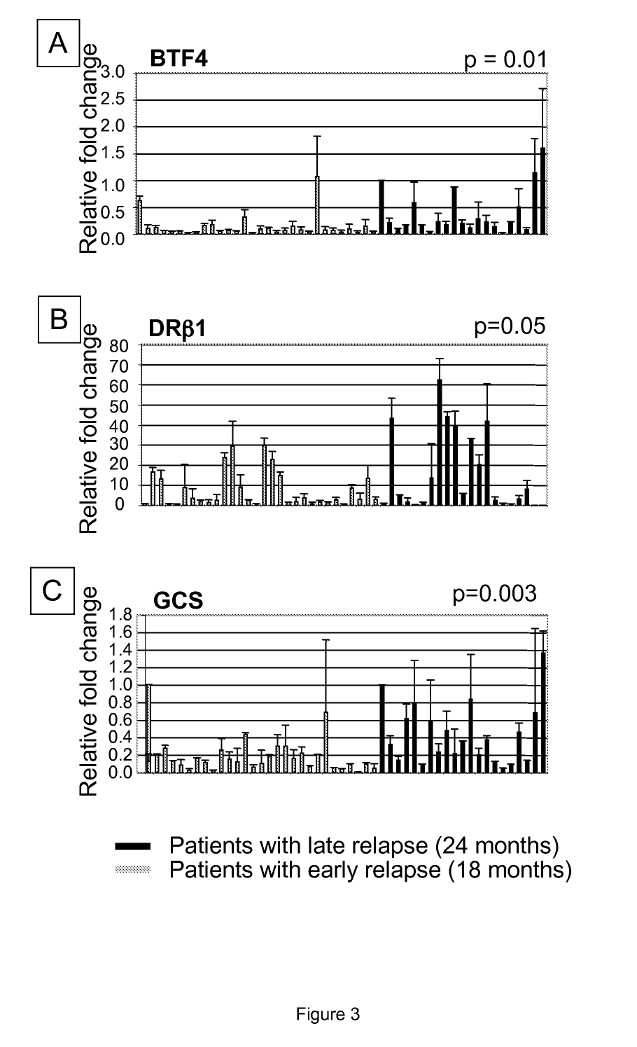

To determine whether the candidate RNA markers identified by microarray and RT-q-PCR may be clinically relevant and reliable to define the prognosis, RNA expression was tested on a large independent set of 41 serous samples. This test set contained 51 samples from 41 patients, of which 26 were associated with early disease progression (less than 18 months) and 15 with late disease progression (greater than two years) (Table 4). Among the candidates tested, only BTF4 (p=0.01), HLA-DRbeta1 (p=0.05) and GCS (p=0.003) showed significant differential expression between the two patient groups (FIG. 3).

### EXAMPLE 5

Association Between RNA Expression and Survival for Patients with Serous Tumors

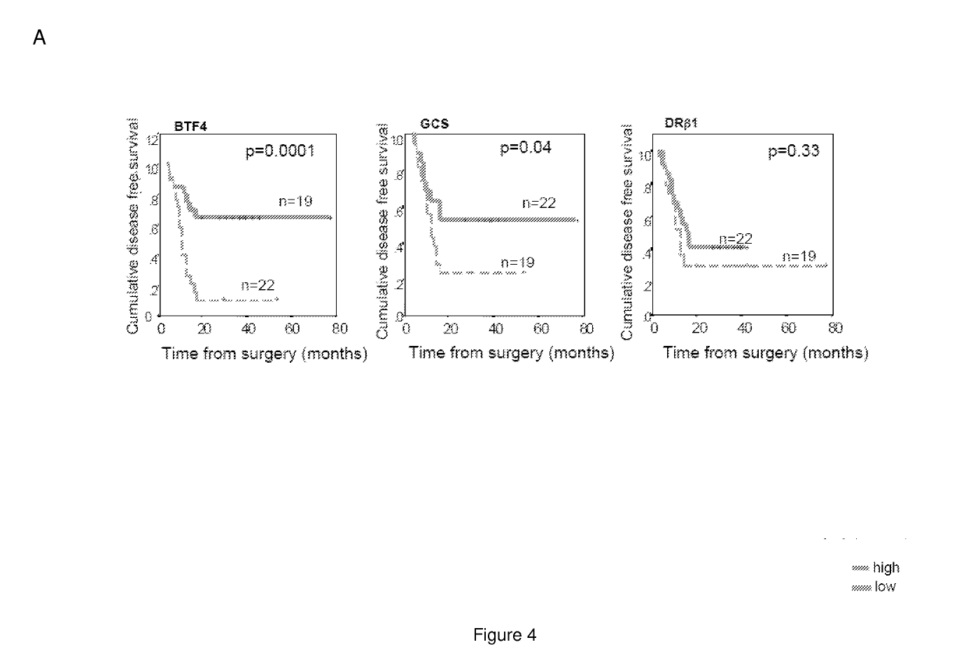

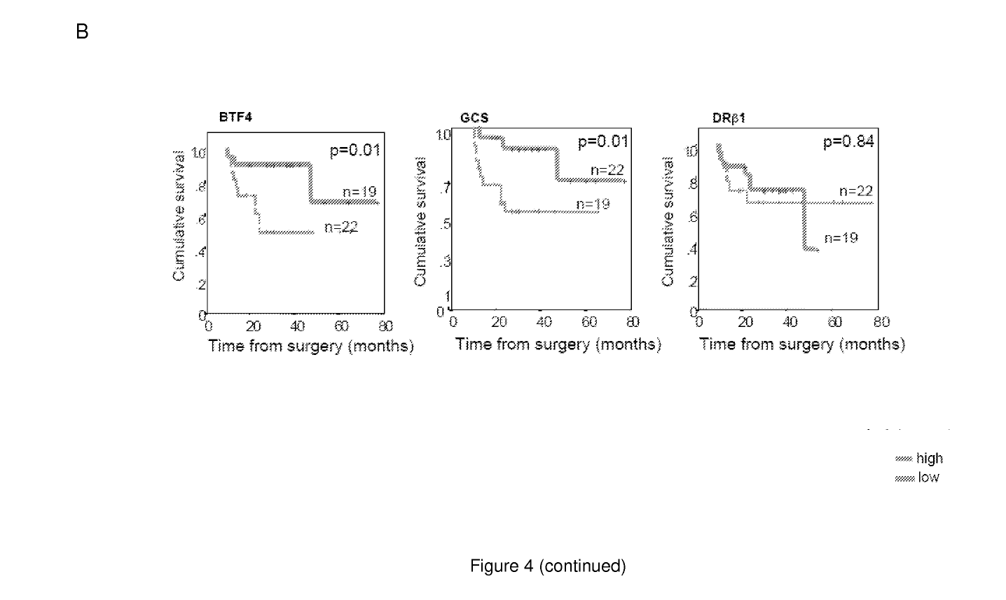

Kaplan-Meier analysis and the Cox proportional hazard model were used to estimate the association between BTF4, HLA-DRbeta1 or GCS expression and disease-free survival (DFS) before 18 months or overall survival (OS) for the 41 patients with serous tumors. The optimal threshold values that could be used for each marker to predict survival and assign labels of early or late disease progression risk was estimated using ROC curves. There was a strong association between BTF4 expression and DFS (p=0.0001, log rank test) or OS (p=0.01, log rank test) (FIG. 4). Mean DFS and OS were 54 months and 64 months, respectively, for patients with high levels of BTF4 expression compared to 11 months and 37 months, respectively, for patients with low levels of BTF4 expression. GCS expression profile also showed a significant association with DFS and OS (p=0.04 and p=0.01 log rank test, respectively) but was weaker than that of BTF4 (Table 4 below and FIG. 4). In contrast, HLA-DRbeta1 expression did not show any significant association with either DFS or OS (p>0.05).

TABLE 4
Prognosis values in serous cohort by Kaplan-Meier analysis
coupled to log rank test and Cox proportional hazard regression model

| | mean | univariate | | multivariate | |
|---|---|---|---|---|---|
| p (log rank) | DFS (months) | p (cox) | HR | p (cox) | HR |

Serous cohort:
41 patients

| | | | | | | | |
|---|---|---|---|---|---|---|---|
| DFS | BTF4 | 0.0001 | 54 vs 11 | 0.0001 | 0.195 | 0.001 | 0.143 |
| | GCS | 0.04 | 45 vs 16 | 0.06 | 0.447 | NS | — |
| | Drβ1 | 0.33 | — | 0.46 | 0.750 | NS | |
| | residual | 0.04 | 22 vs 26 | 0.05 | 2.271 | NS | — |
| | disease | | | | | | |
| | stage | 0.12 | — | 0.02 | 3.202 | NS | — |
| | grade | 0.83 | — | 0.835 | 1.096 | NS | — |
| | age | NA | — | 0.842 | 0.996 | NS | — |
| survival | BTF4 | 0.01 | 64 vs 37 | 0.03 | 0.225 | 0.03 | 0.189 |
| | GCS | 0.01 | 65 vs 31 | 0.03 | 0.181 | 0.03 | 0.178 |
| | Drβ1 | 0.84 | — | 0.85 | 0.896 | NS | — |
| | residual | 0.03 | 41 vs 44 | 0.04 | 3.823 | NS | — |
| | disease | | | | | | |
| | stage | 0.63 | — | 0.21 | 2.222 | NS | — |
| | grade | 0.07 | 48 vs 56 | 0.11 | 5.408 | NS | — |
| | age | NA | — | 0.397 | 1.025 | NS | — |

Non-serous cohort: 18 patients

| | | | | | | | |
|---|---|---|---|---|---|---|---|
| DFS | BTF4 | 0.02 | 35 vs 15 | 0.03 | 0.234 | NS | — |
| | residual | 0.0004 | 7 vs 55 | 0.003 | 9.133 | 0.013 | 6.73 |
| | disease | | | | | | |
| | stage | 0.16 | — | 0.12 | 2.401 | NS | — |
| | grade | 0.33 | — | 0.20 | 1.72 | NS | — |
| survival | BTF4 | 0.003 | 65 vs 23 | 0.02 | 0.076 | 0.03 | 12.20 |
| | residual | 0.0005 | 23 vs 83 | 0.03 | 11.608 | NS | — |
| | disease | | | | | | |
| | stage | 0.01 | 42 vs — | 0.34 | 7.767 | NS | — |
| | grade | 0.28 | — | 0.17 | 1.875 | NS | — |

HR: hazard ratio.
DFS: disease free survival.
NS: non significant.
NA: not applicable. 95% CI.

In univariate and multivariate Cox regression analyses, several clinical prognostic factors such as residual disease, stage, grade and age were evaluated, in relation to BFT4, GCS and HLA-DRbeta1 expression. Low BTF4 expression showed the highest hazard ratio (HR) for DFS (HR=0.195, 95% confidence interval (CI), p=0.0001) whereas low GCS expression showed the highest hazard risk for OS (HR=0.181, 1.21-25, 95% CI, p=0.03) (Table 4). Thus, subjects having a high BTF4 and/or GCS expression are approximately 5 times less likely to relapse within 18 months from surgery than subjects having a low BTF4 and/or expression. In the multivariate analysis, only BTF4 remained an independent variable of prediction with a high risk of progression (HR=0.143, 95% CI, p=0.001) whereas both BTF4 and GCS remained independent variables for prediction of death (HR=0.189, 95% CI, p=0.03 and HR=0.178, 95% CI, p=0.03, respectively). Due to the lack of statistical significance, survival analysis in association with HLA-DRbeta1 expression was not performed.

### EXAMPLE 6

### Performance of BTF4 and GCS in Patient Survival Prediction

To determine the clinical performance of BTF4 and GCS gene expression to predict the DFS and OS in our serous ovarian patient cohort, the sensitivity and specificity of both candidates were evaluated using the label assigned previously from the ROC analysis. Sensitivity for DFS (the fraction of patients correctly diagnosed with ovarian cancer progressing within 18 months) was 78% for BTF4 and 84% for GCS (Table 5). Specificity for DFS (the fraction of patients correctly diagnosed with no disease progression within 18 months after surgery) was 86% and 69% for BTF4 and GCS, respectively. Two independent experiments estimated the reproducibility of the test at 96% for BTF4 and 85% for GCS (Table 5). The combination of two or more candidates identified in this study did not improve the prognostic value of BTF4 (data not shown). The efficiency of the markers to predict OS was lower than for DFS. BTF4 showed 61% sensitivity and 77% specificity. GCS was not able to predict OS significantly.

To evaluate the clinical potential of the markers, their variability of expression was also determined within independent EOC samples obtained from initial surgeries from the same patients. For nine patients, of whom five had early disease progression and four had late progression, two or three samples were available from either the omentum, right or left ovaries. Among these nine patients, the BTF4 markers showed a high concordance in prognostic prediction, with only a single contradictory assignation in one patient and this result was reproducible (analysis conducted in two independent assays). However, this patient was also the only one among the nine with early disease progression (12 months) and a long-term survival (4 years). In contrast, the GCS marker was less robust as independent samples assigned three patients out of nine to separate prognostic outcomes, suggesting that GCS is more physiologically variable and/or more sensitive to the source of sampling than BTF4.

TABLE 5
Performance of individual markers as predictors of
disease-free survival (DFS) or overall survival (OS)

| | | | BTF4 | GCS | DRbeta1 |
|---|---|---|---|---|---|
| DFS | serous | sensitivity | 19.5/25 (78% +/− 0.7%) | 21/25 (84% +/− 2.8%) | — |
| | | specificity | 14/16 (86% +/− 0%) | 11/16 (69% +/− 0%) | — |
| | | reproducibility | 96% | 85% | — |
| | non- | sensitivity | 5/8 (63% +/− 0%) | — | 6/8 (75% +/− 0%) |
| | serous | specificity | 8.5/10 (85% +/− 0.5%) | — | 6.5/10 (65% +/− 0.7%) |
| | | reproducibility | 95% | — | 95% |
| OS | serous | sensitivity | 17/28 (61% +/− 0.5%) | — | — |
| | | specificity | 10/13 (77% +/− %) | — | — |
| | | reproducibility | 96% | — | — |
| | non- | sensitivity | 4/6 (67% +/− 0%) | — | 5/6 (83% +/− 0%) |
| | serous | specificity | 8.5/12 (71% +/− 0.5%) | — | 6.5/12 (54% +/− 0.5%) |
| | | reproducibility | 95% | — | 95% |

### EXAMPLE 7

### Validation of Candidate Genes with Samples of Different Histopathology Types

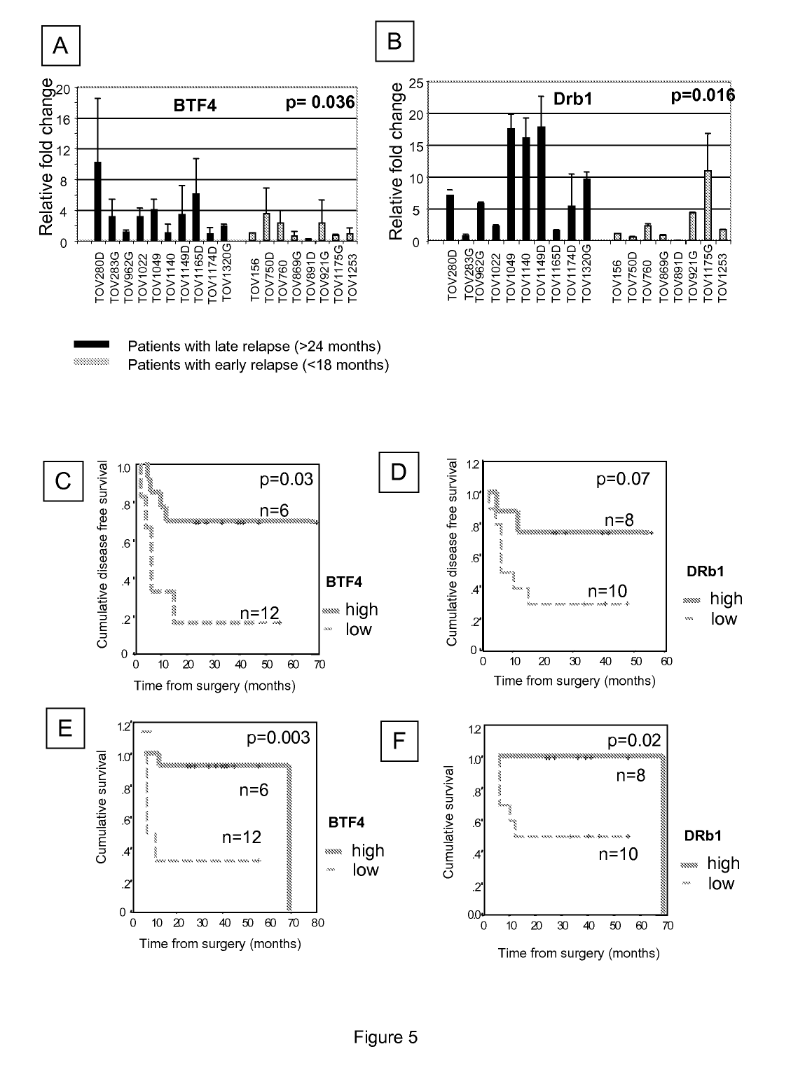

BTF4, GCS and HLA-DRbeta1 expressions were also evaluated as markers to define the prognosis of patients with tumors with less common histopathological subtypes such as non-serous clear cells and endometrial subtypes. RT-q-PCR analyses were performed on 18 patients with early or late disease progression. BTF4 (p=0.036, t-test) and HLA-DRbeta1 (p=0.016, t-test) showed statistically significant differential expression, whereas GCS did not (p=0.171, t-test, data not shown) (FIG. 5). Kaplan-Meier analysis and Cox regression model confirmed the association of BTF4 expression with DFS and OS in the non-serous patient cohort (Table 6, FIGS. 5C and E). BTF4 remained an independent variable only for the prediction of OS (p=0.003, Table 6). The sensitivity and specificity of BTF4 expression to predict DFS before 18 months was also lower for the non-serous patients (63% and 85%, respectively) than for serous patients (Tables 5 and 6). However, this sensitivity and specificity remained similar for the prediction of OS in the serous and non-serous patient cohorts (67% and 71%, respectively). HLA-DRbeta1 expression exhibited better sensitivity than BTF4 expression to predict DFS or OS in non-serous patients (75% vs 63% and 83% vs 67%, respectively), but a lower specificity (65% vs 85% and 54% vs 71%, respectively). As GCS did not show significant differential expression in RT-q-PCR analysis, survival analyses were not performed for this candidate in the non-serous set.

TABLE 6
Prognosis values in clear cells and endometroid cohort by Kaplan-Meier
analysis coupled to log rank test and Cox proportional hazard regression model

| | | | mean | | | | |
|---|---|---|---|---|---|---|---|
| | | | DFS | univariate | | multivariate | |
| | | p (log rank) | (months) | p (cox) | HR | p (cox) | |
| DFS | BTF4 | 0.02 | 35 vs 15 | 0.03 | 4.274 | NS | — |
| | DRbeta1 | NS | | 0.14 | 3.268 | NS | — |
| | GCS | NS | | 0.23 | 1.597 | NS | — |
| | residual | 0.0004 | 7 vs 55 | 0.003 | 9.133 | 0.013 | 6.734 |
| | disease | | | | | | |
| | stage | 0.16 | — | 0.12 | 2.401 | NS | — |
| | grade | 0.33 | — | 0.2 | 1.72 | NS | — |
| SURVIVAL | BTF4 | 0.003 | 65 vs 23 | 0.02 | 13.156 | 0.03 | 0.082 |
| | DRbeta1 | 0.02 | 69 vs 34 | 0.27 | 58.823 | NS | — |
| | GCS | NS | | 0.61 | 1.125 | NS | — |
| | residual | 0.0005 | 23 vs 83 | 0.03 | 11.608 | NS | — |
| | disease | | | | | | |
| | stage | 0.01 | 42 vs — | 0.34 | 7.767 | NS | — |
| | grade | 0.28 | — | 0.17 | 1.875 | NS | — |

HR = hazard ratio.
DFS = disease-free survival.
NS = non significant

### EXAMPLE 8

### Allele-Specific Expression of BTF4

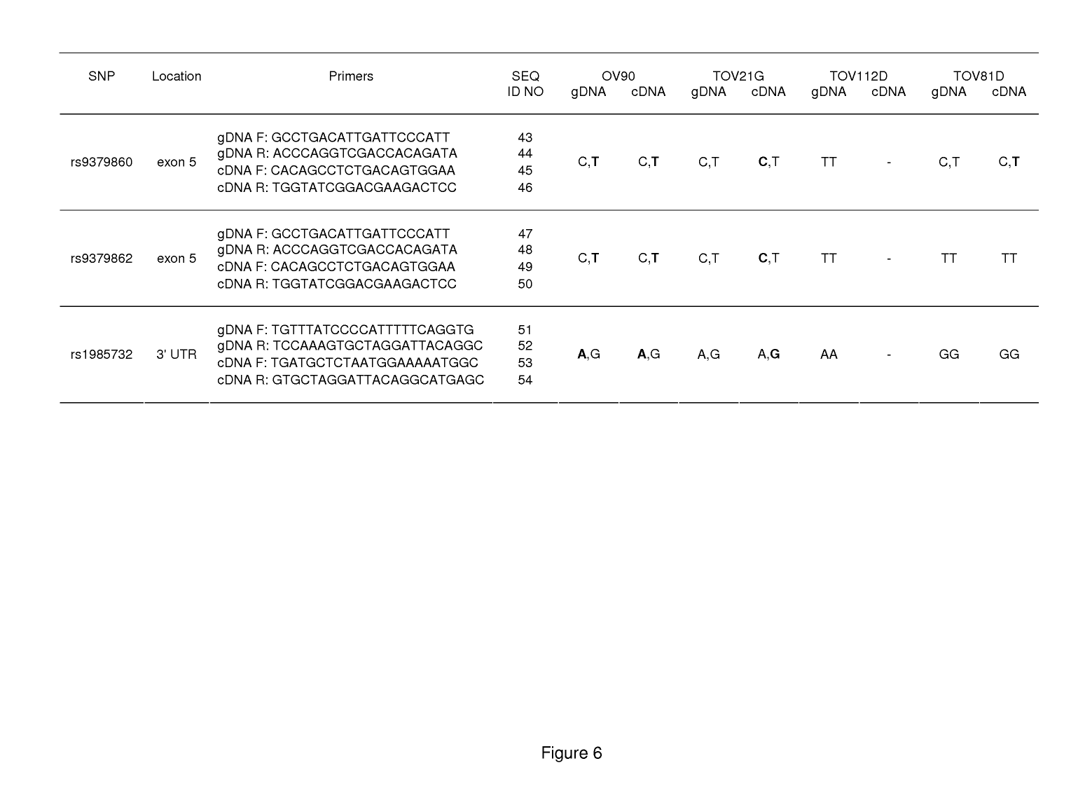

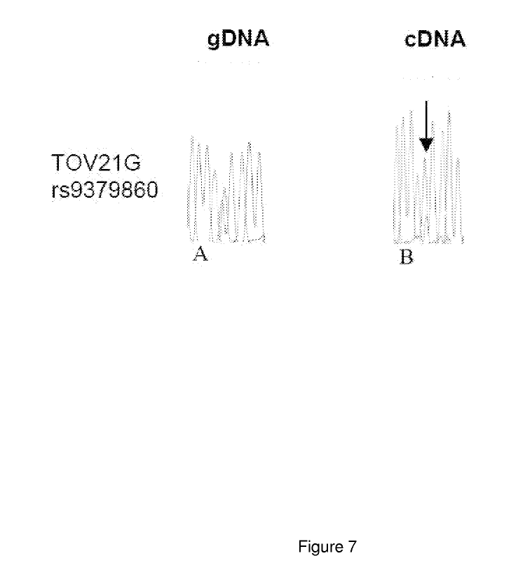

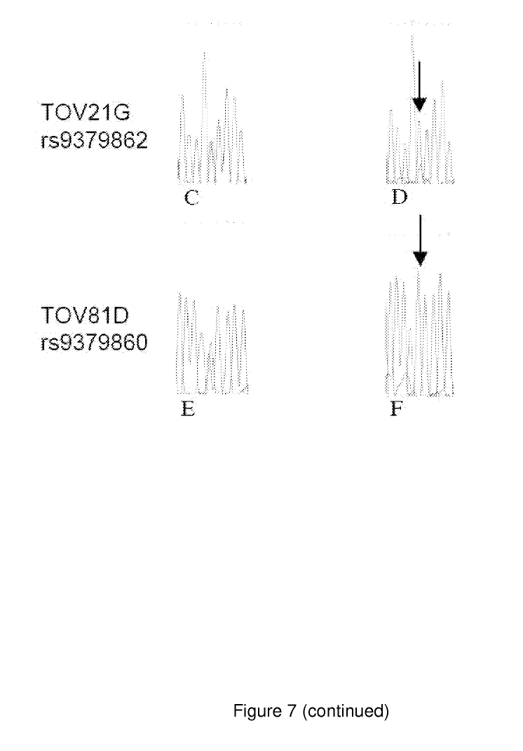

BTF4 is part of an emerging list of autosomal genes exhibiting allelic expression (4). The notion that allelic expression may also be present in EOC tissues is reinforced by the observation that two EOC cell lines heterozygous for BTF4, exhibited deviations from the expected 50:50 allele ratio in the analysis of gene expression (FIGS. 6 and 7). Allele-specific expression of BTF4 was established by sequencing genomic DNA and corresponding cDNA from EOC cell lines (OV90, TOV21G, TOV112D and TOV81D) representing regions that contained possible single nucleotide polymorphisms (SNPs) based on the Human Genome Browser. cDNA expression was not detectable for TOV112D (−). For the TOV21G cell line, the C allele was predominant in SNPs rs9379860 and rs9379862 while the G allele was in excess for the rs1985732 SNP. For the TOV81D cell line, the T allele was predominant for the rs9379860 SNP. As for the OV90 cell line, it is impossible to conclude whether there is differential expression of certain SNP as sequencing of the gDNA suggests an imbalance of genomic alleles.

### EXAMPLE 9

### Detection of the BTF4 Protein

TOV112D or TOV1946 cells were transiently transfected for 48 hr with plasmids containing gene encoding for BT3.1 or BTF4/BT3.2 and the proteins were detected using an anti-CD277 antibody which is specific for both BT3.1 and BTF4.1. Then, cells were lysed and total extracts were submitted to western-blotting on acrylamide gel (10%) and transferred on nitrocellulose membrane. The membrane was hybridized with anti-RT3.1 (anti-CD277 eBioscience), dilution 1/250, and after 2 washes, the membrane was re-hybridized with an anti-IgG1 antibody (Santa Cruz), dilution 1/5000, for 45 nm. Loading control was assessed with anti-GAPDH antibody.

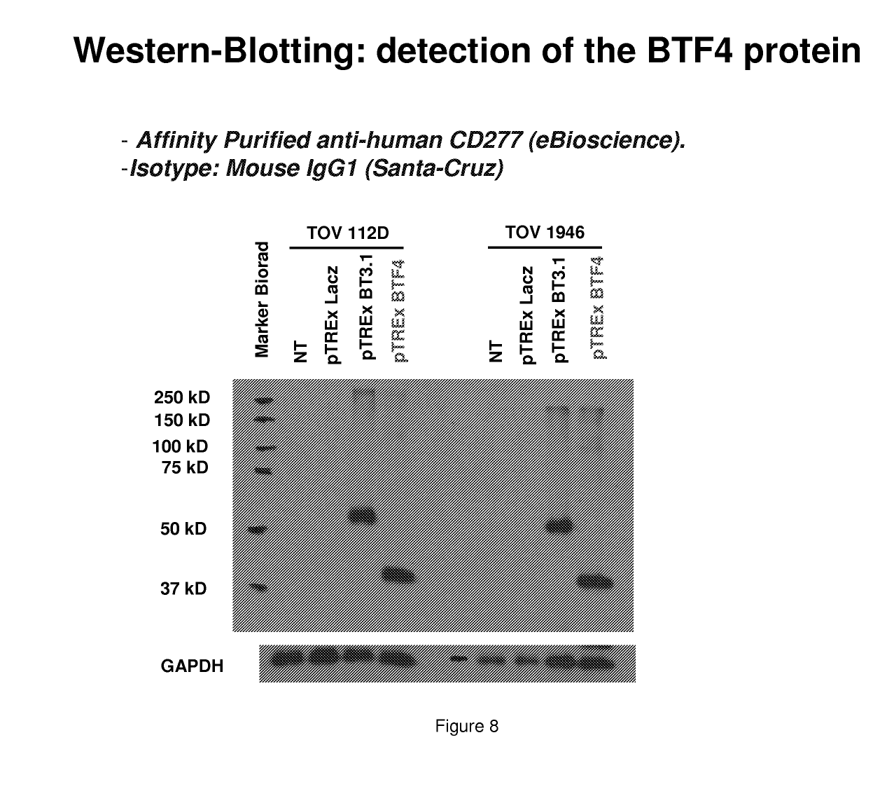

As can be seen on FIG. 8, BTF4 protein could be detected in both cell lines using the anti-human CD277 antibody. This antibody is able to detect all three members of the BT3 family (i.e., BT3.1; BT3.2 and BT3.3 (BTF4)) which share 95% identity at the mRNA level. Methods of the present invention preferably use antibodies which are specific to BTF4 such as commercially available BTF4 antibodies from antibodies from Strategic Diagnostics and from Novus Biological. As the protein sequence of all three members is known, one skilled in the art could identify the regions in BTF4 which differ from the other members and use these fragments to generate BTF4 antibodies using methods which are routine in the art.

The invention being hereinabove described, it will be obvious that the same be varied in many ways. Those skilled in the art recognize that other and further changes and modifications may be made thereto without departing from the spirit of the invention, and it is intended that all such changes and modifications fall within the scope of the invention, as defined in the appended claims.

### REFERENCES

1. Auersperg, N., Wong, A. S., Choi, K. C., Kang, S. K., and Leung, P. C. Ovarian surface epithelium: biology, endocrinology, and pathology. Endocr Rev, 22: 255-288., 2001. Agarwal, P., Bagga, R., Jain, V., Kalra, J. & Gopalan, S. Familial recurrent molar pregnancy: a case report. Acta Obstet Gynecol Scand 83, 213-4 (2004).
2. Serov, S. F., Scully, R., and Sobin, L. H. Histological typing of ovarian tumours., Vol. 9. Geneva: World Health Organization, 1973.
3. Chuaqui, R. F., Cole, K. A., Emmert-Buck, M. R., and Merino, M. J. Histopathology and molecular biology of ovarian epithelial tumors. Ann Diagn Pathol, 2: 195-207, 1998.
4. Pastinen T, Sladek R, Gurd S, et al. A survey of genetic and epigenetic variation affecting human gene expression. Physiol. Genomics 2004; 16:184-193.
5. Therasse P, Arbuck S G, Eisenhauer E A, et al. New guidelines to evaluate the response to treatment in solid tumors. European Organization for Research and Treatment of Cancer, National Cancer Institute of the United States, National Cancer Institute of Canada. Natl Cancer Inst 2000; 92:205-16.)
6. Adam B L, Qu Y, Davis J W, et al. Serum protein fingerprinting coupled with a pattern-matching algorithm distinguishes prostate cancer from benign prostate hyperplasia and healthy men. Cancer Res 2002; 62:3609-14.
7. Ouellet V, Provencher D M, Maugard C M, et al. Discrimination between serous low malignant potential and invasive epithelial ovarian tumors using molecular profiling. Oncogene 2005; 24:4672-87.
8. Le Page C, Ouellet V, Madore J, et al. Gene expression profiling of primary cultures of ovarian epithelial cells identifies novel molecular classifiers of ovarian cancer. Br J Cancer 2006; 94:436-45.
9. Olivier R I, van Beurden M, Van T V L J. The role of gene expression profiling in the clinical management of ovarian cancer. Eur J Cancer 2006; 42:2930-8.
10. Mian S, Ugurel S, Parkinson E, et al. Serum proteomic fingerprinting discriminates between clinical stages and predicts disease progression in melanoma pateints. J. Clin. Oncol., 2005; 23:5088-5093.

1. A method for prognosing the risk of early ovarian cancer relapse in a subject having ovarian cancer comprising:
a) detecting the level of at least one marker selected from the group consisting of BTF4; GCS and HLA-DRbeta1 in a sample from said subject; and
b) comparing the level of said at least one marker with that of a corresponding control sample,
wherein the detection of a lower level of said at least one marker compared to that in the corresponding control sample is indicative that the subject is at risk of early cancer relapse.

2. The method of claim 1, wherein said ovarian cancer is epithelial ovarian cancer.

3. The method of claim 2, wherein said ovarian cancer is serous epithelial ovarian cancer.

4. The method of claim 1, wherein said sample is an ovarian biopsy.

5. The method of claim 2, wherein said level is an mRNA level.

6. The method of claim 5, wherein said marker is BTF4.

7. The method of claim 5, wherein said marker is GCS.

8. The method of claim 3, wherein said marker is BTF4 or GCS.

9. The method of claim 1, wherein said ovarian cancer is serous epithelial ovarian cancer and said marker is BTF4 or HLA-DRbeta1.

10. The method of claim 2, wherein said subject had undergone cytoreductive surgery to remove cancer cells.

11. The method of claim 10, further comprising the step of selecting a treatment in light of the results of step b).

12. The method of claim 8, wherein said subject has received as a first line chemotherapy treatment a combination of platinum-based and taxan-based chemotherapy.

13. The method of claim 8, wherein said subject has received as a first line chemotherapy treatment a treatment selected from the group consisting of (i) carboplatin alone; (ii) carboplatin in combination with paclitaxel or docetaxel; (iii) cisplatin alone; and (iv) cisplatin in combination with paclitaxel or docetaxel.

14. A method of stratifying a subject having ovarian cancer comprising:
a) detecting the level of at least one marker selected from the group consisting of BTF4; GCS and HLA-DRbeta1; and
b) comparing the level of said marker with that of a corresponding control sample,
whereby the results of the detecting step enable the stratification of the subject having ovarian cancer as belonging to a subclass of ovarian cancer.

15. The method of claim 14, wherein said subclass is selected from:
i) early cancer relapse; and
ii) late cancer relapse or no cancer relapse.

16. The method of claim 15, wherein the detection of a lower level of BTF4 or GCS compared to that in the corresponding control sample is indicative that the subject is at risk of early cancer relapse.

17. The method of claim 16, further comprising the step of selecting a treatment in light of the results of step b).

18. The method of claim 17, wherein said cancer is epithelial ovarian cancer.

19. The method of claim 18, wherein, when said subject having ovarian cancer belongs to the early cancer relapse subclass, an aggressive first line chemotherapy treatment is selected.

20. The method of claim 19, wherein said aggressive first line chemotherapy treatment is intraperitoneal chemotherapy.

21. The method of claim 20, wherein said level is an mRNA level.

22. The method of claim 21, wherein said marker is BTF4.

23. The method of claim 32, wherein said therapy is a combination of carboplatin and paclitaxel.

24. A kit to prognose the risk of early ovarian cancer relapse in a subject diagnosed with ovarian cancer comprising means for determining the expression level of at least one marker selected from the group consisting of BTF4, GCS and HLA-DRbeta1 in a sample from said subject together with instructions for prognosing the risk of early ovarian cancer relapse.
